(19) United States
(12) Patent Application Publication
(10) Pub. No.: US 2004/0229814 A1
(43) Pub. Date: Nov. 18, 2004

(54) Methods and compositions for protecting against cataract development associated with vitrectomies

(76) Inventor: James Dillon, New York, NY (US)

Correspondence Address: BROWN RAYSMAN MILLSTEIN FELDER & STEINER LLP, 163 MADISON AVENUE, P O BOX 1989, MORRISTOWN, NJ, 07962-1989, US

(21) Appl. No.: 10/650,357

(22) Filed: Aug. 27, 2003

Related U.S. Application Data
(60) Provisional application No. 60/406,907, filed on Aug. 28, 2002.

(51) Int. Cl. A61K 38/05; A61K 48/00

(52) U.S. Cl. 514/18; 514/44

(57) ABSTRACT

The present invention provides methods and compositions for protecting against cataract development during and after a vitreous replacement, and for treating cataracts in a subject. The methods include using, for vitreous replacement in a vitrectomy, a vitreous replacement solution having a lower oxygen concentration than an air-saturated vitreous replacement solution. The compositions include low-oxygen-concentration vitreous replacement solutions, which may comprise reduced glutathione and/or ascorbic acid. Also provided are methods of using the compositions, during a vitreous replacement or vitrectomy, to protect against cataract development in a subject.

### RELATED APPLICATION

This application claims the benefit of U.S. Provisional Application No. 60/406,907, filed Aug. 28, 2002.

### STATEMENT OF GOVERNMENT INTEREST

This invention was made with government support under NIH Grant No. R01 EY02283. As such, the United States government has certain rights in this invention.

### COPYRIGHT NOTICE

A portion of the disclosure of this patent document contains material that is subject to copyright protection. The copyright owner has no objection to the facsimile reproduction by anyone of the patent document or the patent disclosure, as it appears in the Patent and Trademark Office patent files or records, but otherwise reserves all copyright rights whatsoever.

### BACKGROUND OF THE INVENTION

1. Field of the Invention

The present invention relates to the field of ophthalmology, and, more particularly, to a method and composition for protecting against cataract development.

2. Description of the Related Art

Cataract development—or the opacification of portions of the eye, including the lens—is one of the major causes of preventable blindness and visual impairment. Cataract formation is a serious problem in developed countries. However, due, in part, to a lack of quality health care, its impact is even greater in less-developed countries, where 90% of the world's visual impairment sufferers are found.

Progress in techniques for slowing, and protecting against, cataract development would be of profound benefit to society. Estimates suggest that, if it were possible to delay the onset of cataract development for 10 years (on average) per sufferer, the quality of life for sufferers would greatly increase, and the worldwide economic burden would be reduced by the vast sum of 5-6 billion dollars annually. However, determination of the causes and mechanisms of cataract development, as well as effective measures to slow or protect against cataract development, both in high-risk subjects and in general, has proved difficult. In fact, there remains much to be learned about aspects of the physiology and chemistry of the eye, and the mechanisms by which overall ocular transparency is maintained.

At present, several factors are known to increase the risk of cataract (including nuclear cataract) development. For example, it is known that cataract development becomes increasingly likely as people age. During aging, the ratio of cholesterol:lipid increases in the lens. In this regard, it has been demonstrated that, in the fluid phase of saturated and unsaturated phosphatidylcholine membranes, the addition of 50 mol % cholesterol can cause a decrease in the oxygen permeability coefficient by a factor of 3-5 (Subczynski et al., Oxygen permeability of phosphatidylcholine-cholesterol membranes. Proc. Natl. Acad. Sci. USA, 86:4474-78, 1989; Subczynski et al., Effect of alkyl chain unsaturation and cholesterol intercalation on oxygen transport in membranes: A pulse ESR spin labeling study. Biochemistry, 30:8578-90, 1991a).

Clinical studies have also shown that nuclear cataracts and other lesions commonly occur following vitrectomy, particularly in patients older than 50 years. The development of nuclear cataracts in patients who have undergone vitrectomy is well documented (Chung et al., Cataract formation after pars plana vitrectomy. Kaohsiung J. Med. Sci., 17:84-89, 2001; Hsuan et al., Posterior subcapsular and nuclear cataract after vitrectomy. J. Cataract Refract. Surg., 27:437-44, 2001), and many (especially older) patients require additional surgery due to this complication. Nuclear cataracts generally arise some time later than (within a year or so of) the associated vitrectomies, and appear to occur to a greater extent in patients in which a tamponade is used. Lens changes have been reported to progress in 41-80% of operated eyes, following removal of idiopathic epiretinal membranes using vitrectomy (Cherfan et al., Nuclear sclerotic cataract after vitrectomy for idiopathic epiretinal membranes causing macular pucker. Am. J. Ophthalmol., 11 1(4):434-38, 1991; Smiddy et al., Vitrectomy for macular traction caused by incomplete vitreous separation. Arch. Ophthalmol., 106(5):624-28, 1988). Sawa et al. (Assessment of nuclear sclerosis after nonvitrectomizing vitreous surgery. Am. J. Ophthalmol., 132(3):356-62, 2001), however, reported no post-surgical lens changes following non-vitrectomyzing surgery for epiretinal membrane removal.

The underlying cause of post-operative nuclear cataracts is still unclear. However, reports suggest that nuclear sclerosis may reflect alterations in the metabolic environment of the lens resulting from removal of the vitreous body. In particular, since the lens is an avascular tissue, and depends on diffusion for its supply of oxygen, changes in oxygen tension may play a key role in post-surgical cataract development.

It is believed that low levels of oxygen in the lens are essential to its normal development and the long-term maintenance of transparency (Eaton, J. W., Is the lens canned? Free Radic. Biol. Med., 11(2):207-13, 1991). This theory is supported by the observation that hyperbaric oxygen treatment can cause nuclear sclerosis of the lens, especially in elderly patients (Palmquist et al., Nuclear cataract and myopia during hyperbaric oxygen therapy. Br. J. Ophthalmol., 68:113-17, 1984). It has also been demonstrated that, when old human lens nuclear protein is exposed to air, superoxide is spontaneously produced (Linetsky et al., Spontaneous generation of superoxide anion by lens proteins and calf lens proteins ascorbylated in vitro. Exp. Eye Res., 69:239-48, 1999). This apparently accounts for the resultant nuclear cataracts developed during hyperbaric oxygen treatments (Palmquist et al., Nuclear cataract and myopia during hyperbaric oxygen therapy. Br. J. Ophthalmol., 68:113-17, 1984). In addition, there appear to be autofluorescence increases in the nucleus that precede opacification (Ogura et al., Quantitative analysis of lens changes after vitrectomy by fluorophotometry. Am. J. Ophthalmol., 111:179-83, 1991; Ogura et al., Prospective longitudinal studies on lens changes after vitrectomy—quantitative assessment by fluorophotometry and refractometry. Nippon Ganka Gakki Zasshi, 97:627-31, 1993), occurring within as little as 3 months following vitrectomy. Prior to the present invention, it was not suggested that both fluorescence and subsequent opacification are due to chemical changes resulting from the introduction of oxygen into the lens environment, and that these changes may occur because the vitreal replacements (e.g., BSS+) are at normal oxygen tensions.

The partial pressure of oxygen in the vitreous body has been measured for many species (Fitch et al., Measurement and manipulation of the partial pressure of oxygen in the rat anterior chamber. Curr. Eye Res., 20(2):121-26, 2000), including the human eye (Sakaue et al., Comparative study of vitreous oxygen tension in human and rabbit eyes. Invest. Ophthalmol. Vis. Sci., 30(9):1933-37, 1989). Most of these measurements were conducted using a polarographic microelectrode, a technique which is complicated by the consumption of oxygen during the measurements—a potential disadvantage in tissues, like the lens, with low oxygen tension. Due to this and other technical difficulties (including the fragility of the glass-coated electrode), only limited information exists concerning lens oxygen levels.

The state of the art regarding the possible role of oxygen in cataract formation, as discussed above, is further described in additional patent and non-patent publications. For example, Obara (The oxidative stress in the cataract development, Nippon Ganka Gakkai Zasshi, 99(12):1303-41, 1995) discusses the effects of oxidation-related substances on the eye, including the lens. Furthermore, Elstner et al. (Biochemical model reactions for cataract research, Ophthalmic Res., 17(5):302-07, 1985) demonstrate that activated oxygen species may induce cataract formation. Varma et al. (Oxidative stress on lens and cataract formation: Role of light and oxygen, Curr. Eye Res., 3(1):35-57, 1984) explain that oxidative stress may, in some instances, participate in cataract formation. Additionally, Helbig et al. (Oxygen in the anterior chamber before and after cataract operation, Ophthalmologie, 92(3):325-28, 1995) discuss changes in oxygen supply to the anterior segment of the eye following cataract surgery, and the possible clinical relevance thereof to ischemically-diseased eyes. Finally, U.S. Pat. No. 5,375,611 discloses compounds for cataract prevention, and U.S. Pat. No. 4,826,872 discloses compounds for cataract treatment.

As stated above, it is also known that anti-oxidants can have beneficial physiological effects, including benefits to the eye, and recent literature and publications reflect this. For example, Patent Cooperation Treaty Publication No. WO 01/64661, published Feb. 23, 2001, discloses the use of certain anti-oxidants in treating oxidative-stress-induced diseases, including cataracts and heart disease. Furthermore, U.S. Pat. No. 5,817,630 discloses the use of glutathione antioxidant drops to alleviate eye discomfort and improve lens pliability.

It has also been recognized in the art that contact lenses may affect oxygen concentrations in eye structures. For example, McLaren et al. (Measuring oxygen tension in the anterior chamber of rabbits, Investigative Ophthalmology and Visual Science, 39(10):1899-909, 1998) discuss the finding that cameral oxygen tension under PMMA contact lenses is significantly lower than that in an uncovered eye. The association between vitrectomies and cataract formation has also been further considered in the art. For example, Ogura et al. (Quantitative analysis of lens changes after vitrectomy by fluorophotometry, Am. J. Ophthalmol., 11 1(2):179-83, 1991) discuss the oxidation of lens proteins during vitrectomies, and consider whether this could be a possible cause of nuclear cataract development following vitrectomies.

In addition, the art has recognized the importance of ophthalmic irrigating solutions, including vitreal replacement solutions and formulations, even though the increased risk of cataracts following vitrectomy has not been remedied. For example, U.S. Pat. No. 5,604,244 discusses irrigating solutions containing a polyamine antagonist for use in preventing excitotoxicity associated with ophthalmic surgery. Haimann et al. (The effect of intraocular irrigating solutions on lens clarity in normal and diabetic rabbits. Am. J. Ophthalmol., 94(5):594-605, 1982) discuss the effect of Balanced Salt Solution (BSS®) and BSS Plus® as irrigating solutions, indicating that BSS Plus® appears to cause fewer undesirable morphological changes to eye structures than does BSS®. Moreover, McDermott et al. (Ophthalmic irrigants: A current review and update, Ophthalmic Surg., 19(10):724-33, October 1988) discuss the importance of irrigating solutions in ophthalmic surgery, the potential negative effects of such solutions on eye structures, and ideal solution characteristics.

It has been known for many years that excessive oxidation can have a deleterious effect on tissues, including the eye. In addition, it is known that anti-oxidants generally have a favorable physiological effect, including benefits to the eye, under certain circumstances. Nevertheless, little is known concerning the levels of oxygen in the lens, the diffusion of oxygen within various portions of the lens, and the relationship between changes in oxygen tension and age and/or environment. For example, despite evidence of an association between aging and cataract development, and evidence of an association between vitrectomies and cataract development, a physiological and chemical explanation for these associations has not heretofore been presented. Thus, there is a lack of understanding of the mechanisms and processes within the eye which contribute to lens clarity, and of the measures which can be taken to protect against cataract development.

In light of the magnitude of the problems caused by cataracts, in terms of both human suffering and financial expense, methods and compositions to protect against the development of cataracts are needed in the art. Accordingly, given the increased risk of cataract development associated with vitrectomies, measures to decrease this risk would constitute an important step forward in the overall fight against cataract development, and are, therefore, needed in the art.

### SUMMARY OF THE INVENTION

The inventor has used a fiber-optic oxygen sensor system (optode) to measure oxygen tension in the rabbit eye before and after surgery, and has determined that changes in oxygen tension do play a role in the development of cataracts after vitrectomy. Accordingly, the present invention provides methods and compositions for protecting against cataract development during a vitreous replacement, and for treating cataracts in a subject.

In one aspect, the invention provides a method for protecting against cataract development in a subject, by using, during a vitreous replacement, a vitreous replacement solution having a low oxygen concentration.

In another aspect, the invention provides a method for protecting against cataract development in a subject, by using, during a vitreous replacement, a vitreous replacement solution from which at least a portion of the oxygen has been removed.

In still another aspect, the invention provides use of a low-oxygen-concentration vitreous replacement solution during a vitrectomy.

Additionally, the invention provides a low-oxygen-concentration vitreous replacement solution, for use in vitrectomies.

Finally, the invention provides a method for protecting against cataract development and/or for treating a cataract in a subject, by reducing oxygen concentration in a vitreous of the subject.

Additional aspects of the present invention will be apparent in view of the description that follows.

### BRIEF DESCRIPTION OF THE FIGURES

The invention is illustrated in the figures of the accompanying drawings, which are meant to be exemplary and not limiting, and in which like references are intended to refer to like or corresponding parts.

FIG. 1A shows the change in Henry's constant with increasing glycerol in water solutions. FIG. 1B depicts actual oxygen concentrations (measured by the Winkler method) and oxygen tension with increasing glycerol in a glycerovwater mixture.

FIG. 2 depicts a simplified graphical representation of a cow eye, including measured oxygen concentrations in regions of the eye.

FIG. 3A depicts typical optode-signal decay rates resulting from relocation of the optode from an air-saturated solution to an argon-saturated solution. FIG. 3B depicts the linear relationship between viscosity and exponential decay rate.

FIG. 4 depicts optode readings indicating oxygen diffusion rates in a calf lens.

FIG. 5 depicts oxygen electrode readings (gradient of pO₂ in lens) as the electrode is inserted into the center of a live rabbit lens, and slowly pushed through using a micromanipulator.

FIG. 6 sets forth optode readings as the optode is inserted and slowly moved through the vitreous of an anaesthetized rabbit eye.

FIG. 7 depicts decrease in oxygen tension over time, as measured using an optode in a euthanized rabbit.

FIG. 8 depicts loss of oxygen over time, as measured by an optode, in a rabbit vitreous, after bubbling 21% oxygen through the vitreous.

FIG. 9A depicts an actual chromatogram trace of a live rabbit vitreous. FIG. 9B depicts an actual chromatogram trace of a rabbit vitreous, 10 min after sacrifice. FIG. 9C depicts an actual chromatogram trace of an isolated rabbit vitreous, 10 min after oxygen was added to the vitreous.

FIG. 10 illustrates equipment for use in experiments to measure oxygen in lenses. The oxygen measurements were performed using a commercially-available fiber-optic oxygen sensor system (FOXY Fiber Optic Oxygen Sensor System, Ocean Optics Inc., USA). The probe was made out of aluminum, with a diameter of 300 μm, and was specifically designed for the experiments by the inventor.

FIG. 11 depicts a modified horizontal diffusion chamber for use in oxygen diffusion experiments.

FIG. 12 depicts optode readings measuring rates of non-steady-state diffusion of oxygen in lens samples.

FIG. 13A illustrates equipment, for use in oxygen measurement experiments, that allows separation of the anterior and posterior portions of a lens. FIG. 13B depicts the equipment of FIG. 13A with certain modifications, including segregated perfusion inlets and outlets, and separate oxygen probes for each chamber.

FIG. 14 shows oxygen measurements (in mmHg) taken in pre-defined positions within the vitreous. The numbers indicate positions within the eye. Before and after the measurements were taken, the probe was calibrated in 21% oxygen at 39° C., to ensure consistency of the measurements.

FIG. 15 provides the results of oxygen tension measurements (pO₂) taken in a control eye, in pre-defined positions within the vitreous, lens, and anterior chamber. The lowest pO₂ within the vitreous is found in the center of the globe, directly behind the lens. There is no significant difference in the measurements of the posterior lens and the anterior central vitreous.

FIG. 16 illustrates the mean oxygen tension within the normal rabbit eye, including standard deviations.

FIG. 17 depicts the decline of oxygen tension within BSS vitreous replacement, directly after vitrectomy. The plateaus at the beginning and the end of the measurement indicate the standardization of the probe in 21% oxygen.

FIG. 18 depicts oxygen tension before and after vitrectomy. Statistically-significant values are indicated by their p-values. Statistical significance was accepted on a level of p<0.05.

### DETAILED DESCRIPTION OF THE INVENTION

Oxygen is believed to be one of the potential causative agents for the development of nuclear cataracts following vitrectomy. As described herein, the inventor has undertaken experiments to determine the partial pressure of oxygen (pO₂) in different compartments of the rabbit eye, and to describe the changes following vitrectomy.

Specifically, 26 rabbits (3.5-5.3 kg) were anaesthetized, and oxygen tension was probed using a fiber-optic oxygen sensor system (optode). A micromanipulator was employed to ascertain the exact position of the probe within the eye. Measurements were taken pre- and post-vitrectomy, at several defined positions within the vitreous, the lens, and the anterior chamber. Follow-up measurements were performed 1-12 weeks after vitrectomy. The contralateral eye served as a control.

In accordance with these methods, it was determined that oxygen tension in the globe is asymmetrical with the lowest pO₂ in the nucleus of the lens (9.4 mmHg±1.2). The region of the lens near the posterior capsule has an oxygen tension close to the values of the vitreous directly behind the posterior capsule (10 mmHg±0.4). The highest pO₂ within the posterior compartment of the eye was measured close to the retinal surface (40-60 mmHg), depending on neighboring large vessels. The tension dropped off rapidly to 20 mmHg, some 0.5 mm from the retina. From that position to the posterior surface of the lens, there was a shallow gradient of decreasing pO₂. Immediately following vitrectomy, the pO₂ in the BSS replacement varied from approximately 90 mmHG to 140 mmHg, and decreased over approximately 30 min to levels that were 2-3 times that of normal vitreous. Two weeks after vitrectomy, the pO₂ values in the lens were 2-3 times as high as in the control eye (p<0.05). In addition, there was no longer a gradient in the vitreous cavity, except close to the retina. Eight weeks after vitrectomy, pO₂ levels in the lens were decreased, but still remained higher than in the normal eye. The pO₂ gradient in the vitreous was no longer detectable.

As discussed above, the lens and the vitreous are avascular tissues which depend on diffusion for their supplies of oxygen. Because oxygen gradients develop due to diffusional transport, the vitreous and lens oxygen supplies can be characterized by the distribution of local pO₂. In agreement with previous reports, the inventor's results show that vitreal pO₂ is significantly higher in the vicinity of the retina, and is low at a position 0.5 mm away from the retina (Alder and Cringle, The effect of the retinal circulation on vitreal oxygen tension. Curr. Eye Res., 4(2):121-29, 1985; Sakaue et al., Comparative study of vitreous oxygen tension in human and rabbit eyes. Invest. Ophthalmol. Vis. Sci., 30(9):1933-37, 1989). In a study on vitreal pO₂ profiles in cats, Buerk et al. (O₂ gradients and countercurrent exchange in the cat vitreous humor near retinal arterioles and venules. Microvasc. Res., 45(2): 134-48, 1993) found that significant oxygen flows from retinal arteries into the vitreous body, but it is curbed by the vitreous in the vitreoretinal interface. In that study, it was assumed that this diffusing oxygen is used by the inner retina, because the authors determined that oxygen flows towards the retina from the pre-retinal vitreous. However, in the inventor's experiment, there was a shallow gradient of decreasing oxygen extending to the posterior of the lens. This may be explained by additional chemical reactions in the vitreous that involve the ascorbic-acid-mediated conversion of oxygen to hydrogen peroxide, as hypothesized by Eaton (Is the lens canned? Free Radic. Biol. Med., 11(2):207-13, 1991).

The inventor describes herein a gradient of decreasing oxygen, from both the anterior and posterior eye, with a minimum of about 9-10 mmHg in the nucleus of the lens. The inventor believes that this is the first report of the level of oxygen tension in the nucleus of the lens, and that this low concentration agrees with the supposition that low oxygen levels are essential to the health of the lens (Eaton, J. W., Is the lens canned? Free Radic. Biol. Med., 11(2):207-13, 1991; Palmquist et al., Nuclear vacuoles in nuclear cataract. Acta. Ophthalmol. (Copenh.), 64(1):63-6, 1984; Schocket et al., Induction of cataracts in mice by exposure to oxygen. Isr. J. Med. Sci., 8(8):1596-601, 1972). The manner in which this is accomplished is not entirely clear. It is of particular interest that the oxygen tension in the center and posterior parts of the lens is similar to that in anterior vitreous body. This implies that vitreal pO₂ stabilizes, or, to some degree, controls, intralental oxygen levels.

The oxygen changes which take place in the vitreous cavity directly after vitrectomy are quite significant. The rapid decrease in oxygen tension, seen within the first 30 min after the operation, may be due to oxygen consumption by the retina. When oxygen consumption and oxygen diffusion into the vitreous cavity (mainly from retinal vessels) reach a balance, the oxygen level remains stable, but at a level higher than it was prior to the operation. The inventor's results show that this increased oxygen tension remains for quite some time—maybe hours or days. During that time, the lens is exposed to relatively high levels of oxygen, which are 2-3 times higher than normal.

After vitrectomy, the vitreous gel is replaced by an irrigating solution, which, in turn, is eventually replaced by the aqueous humor. Stefansson et al. (Vitrectomy prevents retinal hypoxia in branch retinal vein occlusion. Invest. Ophthalmol. Vis. Sci., 31(2):284-89, 1990) has suggested that this more fluid material will allow for a more even distribution of oxygen. The inventor's data show that there is no longer an oxygen gradient in the vitreous cavity after vitrectomy: oxygen distribution is constant throughout the vitreous cavity (except close to the retina) and the lens. Compared with the normal eye, oxygen tension is significantly higher in the vitreous cavity, especially in the anterior part, which is, in turn, manifested by an increase in oxygen in the lens.

The inventor has hypothesized that post-operative nuclear cataract formation after vitrectomy may be the result of an increase in oxygen tension in the lens. This hypothesis is based on the fact that the lens oxygen environment is significantly changed after vitrectomy. In the normal rabbit eye, oxygen tension is highest in the anterior chamber and directly on the retina, and decreases to a minimum of approximately 9-10 mmHg in the nucleus of the lens. This low level of oxygen seems to be maintained by the metabolism of the anterior lens and the equivalent low pO₂ levels in the adjacent anterior vitreous. Following vitrectomy, the vitreous body is usually replaced by irrigation solution (BSS), gas, air, or silicon oil. All of these replacements contain much higher pO₂ levels than the normal vitreous. Considering that the irrigation solution during surgery is equilibrated with air at an atmospheric pressure, oxygen tension in the irrigation solution is estimated to be about 150 mmHg. Therefore, the lens is exposed to an extremely high level of oxygen during, and shortly after, vitrectomy. Although the irrigation solution is eventually replaced over time, oxygen may diffuse into the lens during this period. In addition, the gradient of decreasing oxygen within the vitreous cavity (posterior to anterior) is permanently disrupted. These changes potentially contribute to nuclear cataract formation following surgery, since oxygen is a known hazard to lens transparency.

Palmquist et al. reported that older patients, who underwent hyperbaric oxygen treatments, developed nuclear cataracts after treatment (Palmquist et al., Nuclear vacuoles in nuclear cataract. Acta. Ophthalmol. (Copenh.), 64(1):63-6, 1986, Palmquist et al., Nuclear cataract and myopia during hyperbaric oxygen therapy. Br. J. Ophthalmol., 68(2): 113-17, 1984). It is also apparent that the increase in oxygen would have a greater effect in the nucleus of older patients—due to an age-related decrease in the anti-oxidant glutathione in the nucleus (Truscott, R. J., Age-related nuclear cataract: a lens transport problem. Ophthalmic. Res., 32(5):185-94, 2000; Ortwerth and Olesen, Glutathione inhibits the glycation and crosslinking of lens proteins by ascorbic acid. Exp. Eye Res., 47(5):737-50, 1988; Shui and Beebe, Oxygen levels in human eyes before and after vitrectomy. Invest. Ophthalmol. Vis. Sci., 44(5):2346, 2003), and the large amount of age-related yellow components that form in the nucleus. In a recent study by Ortwerth et al. (Studies on singlet oxygen formation and UVA light-mediated photobleaching of the yellow chromophores in human lenses. Exp. Eye Res., 74(2):217-29, 2002), protein from the nucleus (but not the cortex) of old human lenses was found spontaneously to form superoxide when exposed to air.

The main thrust of the inventor's study is that increases in oxygen after vitrectomy lead to increases in lenticular oxygen tension, with the subsequent formation of a nuclear cataract. It seems clear from this study that the lens equilibrates with vitreal or “vitreal replacement” oxygen over time, increasing to 2-3 times after 2 weeks, and maintaining a 20% increase in the nucleus for at least 8 weeks. Preservation of the low oxygen environment of the lens, during and after vitrectomy, may also prevent post-operative cataract formation in the future.

In view of the results presented herein by the inventor, the present invention is based on the surprising discovery that levels (concentrations) of oxygen (O₂) in and around the lens of the eye result from diffusion of oxygen from the surrounding regions (specifically, the vitreous and the aqueous), and that oxygen levels in and around the lens are, therefore, dependent, at least in part, on vitreous and aqueous oxygen levels. Low levels of oxygen in certain portions of the eye, including the vitreous (a viscous portion of the eye) and the lens, are very important in preventing cataract development. It has been found that normal vitreous oxygen levels are lower than would be expected. It has also been determined that non-steady-state oxygen diffusion out of a vitreous with higher-than-normal oxygen concentration levels is slower than would be expected. In addition, it has been discovered that the lens is more viscous than was previously estimated.

The present invention is also based on the important discovery that diffusion is not the only mechanism by which oxygen levels in the vitreous are reduced or maintained at low levels; rather, chemical processes in the vitreous, facilitated by enzymes and other substances in the vitreous, can also metabolize and eliminate oxygen in the vitreous or lens. For example, ascorbic acid, a particular anti-oxidant, is utilized in, and is important in facilitating, this metabolism and elimination of oxygen in the lens. Importantly, age causes less-efficient lens enzyme activity, and, therefore, less-efficient lens oxygen metabolism. Despite this, however, diffusion of oxygen from the vitreous into the lens is constant as aging takes place. Thus, oxygen levels in the lens tend to increase as aging takes place. This can at least partially explain, for example, yellowing in the eyes of aging people, and, importantly, increased cataract development risk as people age.

To elaborate, the lens acquires oxygen as a result of oxygen diffusion from the vitreous and the aqueous. The lens metabolizes some of this oxygen for energy. As mentioned above, however, lens oxygen metabolism becomes less efficient and slows down with age. As such, while oxygen diffusion from the vitreous and aqueous does not change with age, oxygen metabolism and consequent elimination from the lens decreases with age. This unbalanced situation results in an increase in lens oxygen levels with age, which appears to result in cataract development.

Significant to the present invention is the observation that low levels of oxygen in portions of the eye, including the vitreous and lens (as compared with typical oxygen levels in other tissues), are important in preserving transparency and preventing cataract development. As a general rule, oxygen is needed in tissues, but high oxygen levels damage tissues. The level of oxygen that is excessive, however, depends on the type of tissue and its normal oxygen level. As discussed further below, the normal oxygen level for the cornea and retina of the eye is essentially the typical tissue level; in contrast, the vitreous and lens are nearly anaerobic.

In view of the foregoing, then, it is clear that oxygen levels in the lens increase significantly after vitrectomy, thereby contributing to cataract formation following surgery. Accordingly, in some embodiments, the present invention generally provides methods and compositions for use in protecting against or treating, cataract development. As used herein, “protecting against cataract development” includes preventing the initiation or start of a cataract, delaying the initiation or start of a cataract, preventing the progression or advancement of a cataract, slowing the progression or advancement of a cataract, and delaying the progression or advancement of a cataract. Cataract development corresponds with lens opacity: increased lens opacity indicates increased cataract development. Therefore, cataract development may be assessed by assessing lens opacity. Lens opacity can be assessed by various methods known in the art, including those disclosed herein. For example, lens opacity can be assessed by a commercially-available Scheimpflug Camera, which is commonly used as a non-invasive means to assess human lens opacity. As further used herein, the term “cataract development” means the initiation or start, progression, or advancement of a cataract.

The methods and compositions of the present invention generally decrease oxygen concentration of a vitreous in a subject, by decreasing oxygen concentration in a vitreous in a subject, or by decreasing oxygen provided to a vitreous of a subject. The subject may be any mammal, but is preferably a human. Unless otherwise stated, the term “oxygen” means O₂. The methods include administration of a dosage of ascorbic acid into the eyes of a subject (e.g., by eye-drop administration of solutions containing ascorbic acid), wherein the dosage is effective to protect against or treat cataracts. The methods further include the use of contact lenses that are semi-permeable to oxygen. Such a lens controls oxygen permeation into an eye of a subject; the oxygen permeates the eye at a rate which is effective in protecting against or treating cataract development, but is sufficient to maintain normal eye metabolism. The methods further include use of eye drops of an optical solution to protect against cataract development, and a method for protecting against cataract development by administering to a subject an optical solution with reduced oxygen concentration (as compared with air-saturated optical solutions). The term “optical solution”, as used herein, is intended to mean any of various solutions for administration to eyes of subjects, including, for example, commercially-available saline solutions for eye-drop administration to subjects.

In other embodiments, the invention generally provides methods and compositions for protecting against cataract development associated with vitrectomies, during a vitreous replacement. The term “vitrectomy” is intended to refer to any of various surgical or other procedures in which all or a portion of a vitreous is removed and replaced with a vitreous replacement substance, such as an ophthalmic irrigating solution (e.g., BSS® or BSS plus®). The methods of the present invention include use of a vitreous replacement solution having a low oxygen concentration, as that term is defined herein. The methods further include using, for vitreous replacement in a vitrectomy, a vitreous replacement solution having an oxygen concentration lower than that of an air-saturated vitreous replacement solution. The methods also include using, for vitreous replacement in a vitrectomy, an initial vitreous replacement solution from which at least a portion of the oxygen resulting from air-saturation of the vitreous replacement solution has been removed. In some embodiments of the present invention, the vitreous replacement solution contains glutathione. Moreover, in some embodiments of the invention, the vitreous replacement solution contains ascorbic acid or a combination of ascorbic acid and reduced glutathione (“GSH”).

In the following description of the embodiments of the invention, reference is made to the accompanying drawings that form a part hereof, and in which is shown, by way of illustration, a specific embodiment in which the invention may be practiced. It is to be understood that other embodiments may be utilized, and structural changes may be made, without departing from the scope of the present invention.

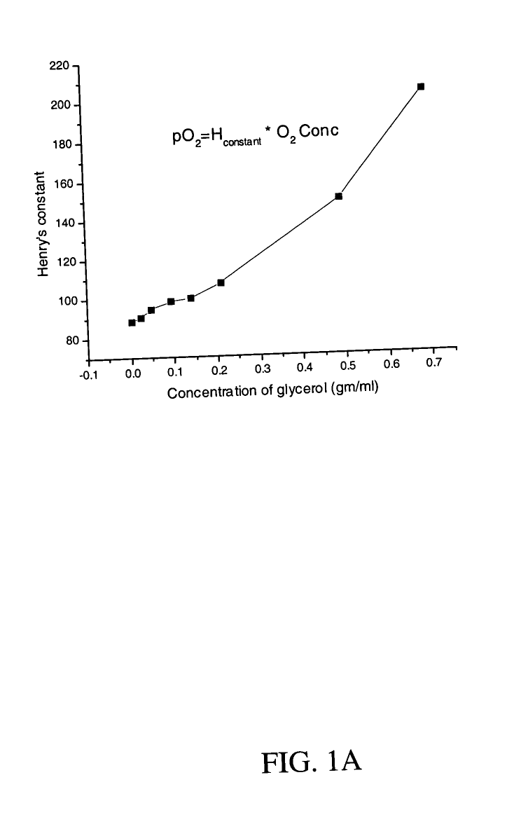

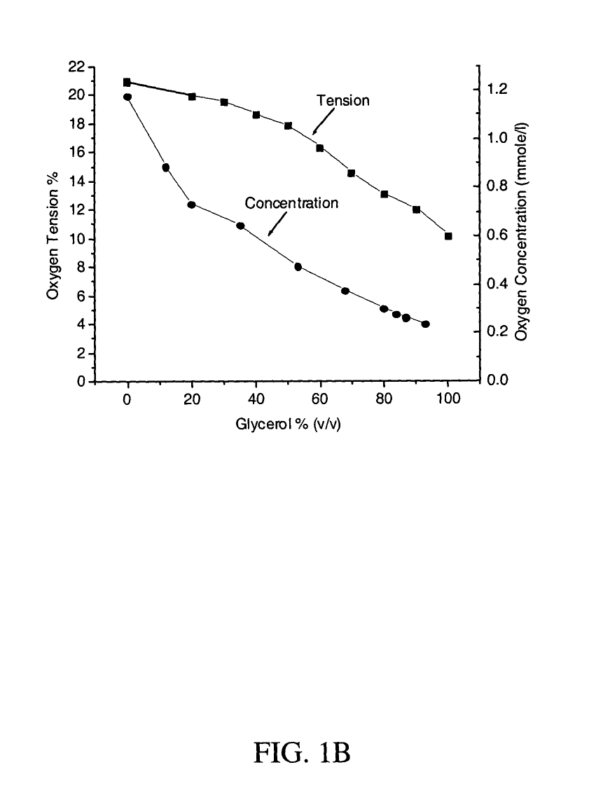

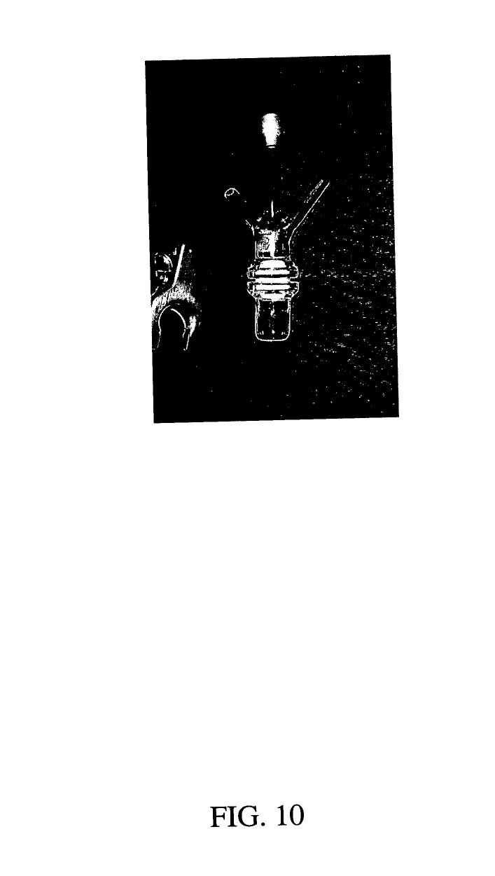

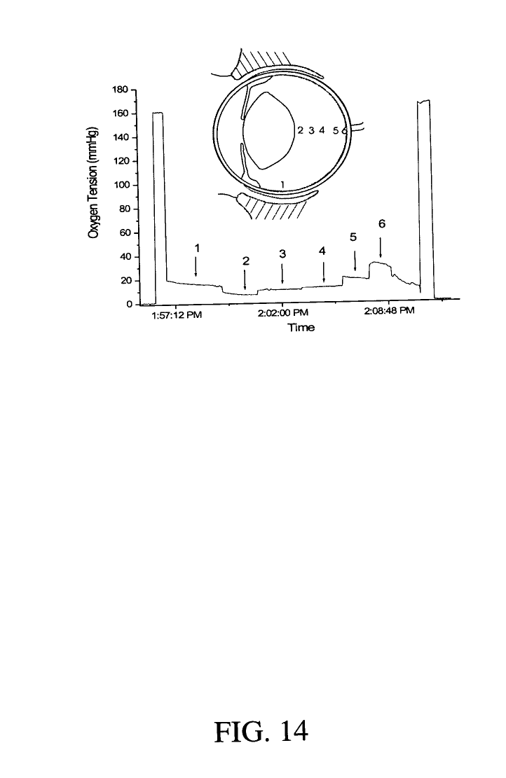

FIGS. 1-9 relate to Examples 1 and 2 below; FIGS. 10-13 relate to Example 3; and FIGS. 14-18 relate to Example 4 below. It should be noted that many of the Examples involve oxygen tension measurement data. “Oxygen tension” is defined as the partial pressure (pO₂) of oxygen gas in a liquid. While oxygen tension, which is relatively easy to measure, is not identical to oxygen concentration, the two are closely related; higher oxygen tension, as would be expected, generally indicates higher oxygen concentration. More details concerning oxygen tension are provided throughout the Examples. Thorough descriptions of the Figures are provided in the Examples; however, the following brief additional commentary is provided.

As noted above, oxygen tension is closely related to oxygen concentration; FIGS. 1A and 1B are used, in part, to explain this relationship.

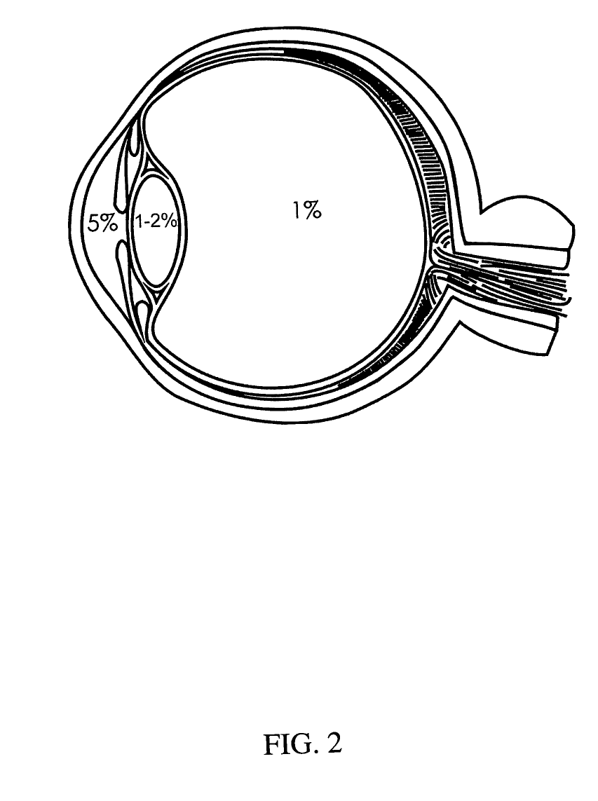

FIG. 2 presents the results of experiments which indicate oxygen concentrations in portions of the eye, including the vitreous. As shown in FIG. 2, the oxygen concentration in the vitreous is indicated to be only 1% or so, which is much less than the 3-5% concentration which might be expected. As such, FIG. 2 is used to provide evidence of the significant observation herein that the level of oxygen in the vitreous is lower than might be expected. This is considered to be evidence of metabolic activity within the vitreous that consumes oxygen, as explained further below.

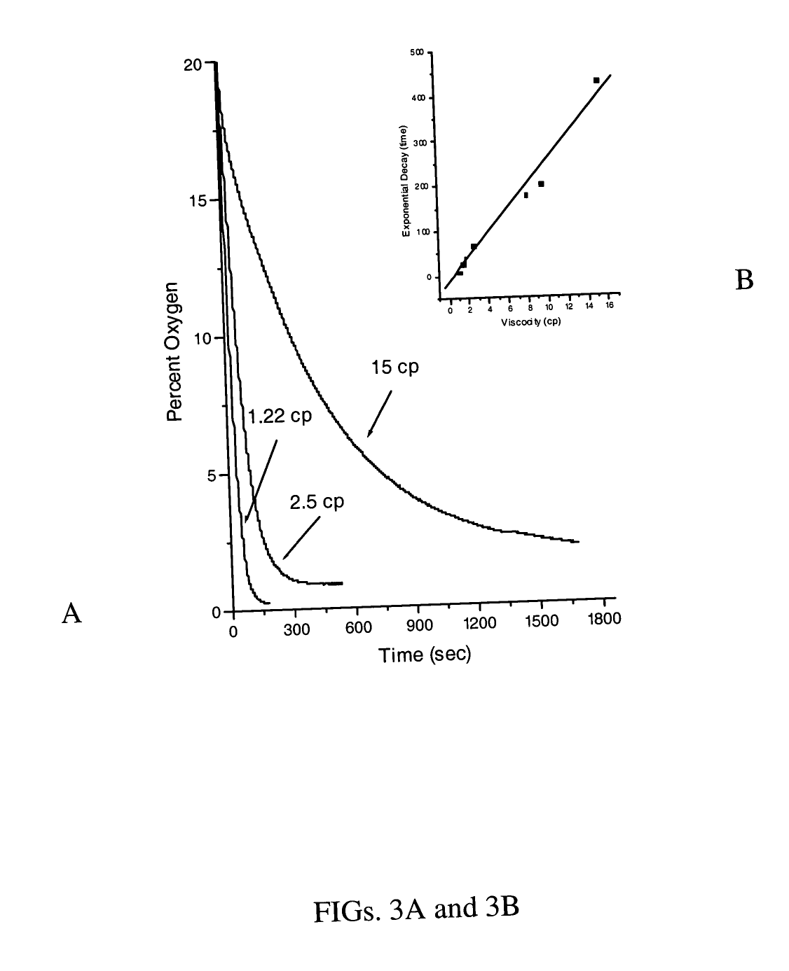

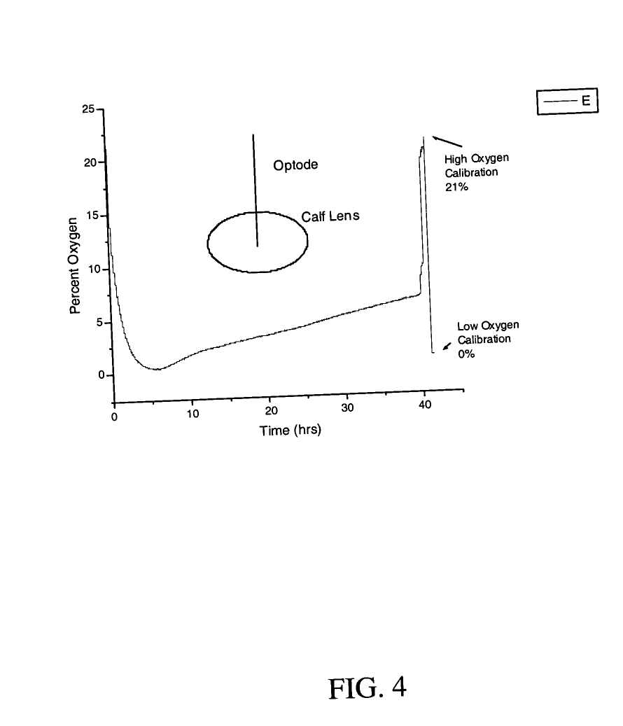

FIGS. 3A and 3B demonstrate the manner in which oxygen diffusion slows as viscosity of the medium increases. Additionally, FIG. 4 shows oxygen levels in various portions of a calf lens. It is noted in the discussion of FIG. 4 that lens viscosity is believed to be greater than has been stated in certain literature, and FIG. 4 provides evidence of the slower rate of oxygen diffusion resulting from the highly viscous vitreous.

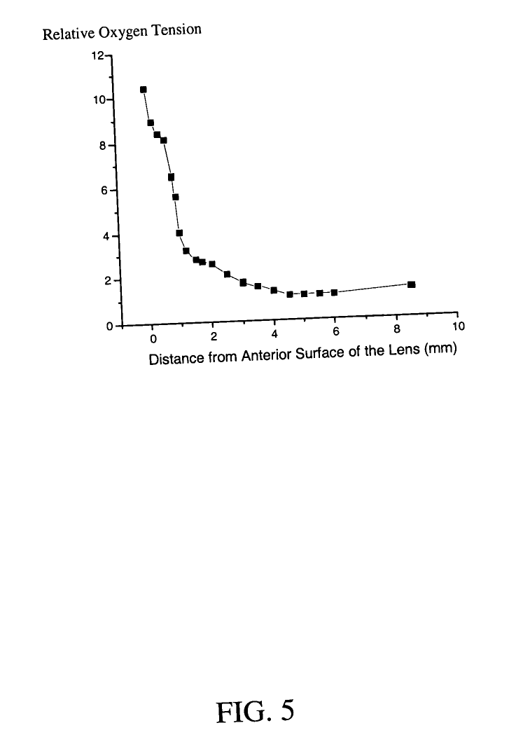

FIG. 5 shows oxygen tensions in different portions of a rabbit lens. The data shown in FIG. 5 indicate that oxygen tension is dramatically less toward the posterior of the lens. It is suggested that the vitreous is at a low oxygen concentration, and contributes to the low oxygen concentration found in the lens, because the posterior of the lens adjoins the vitreous.

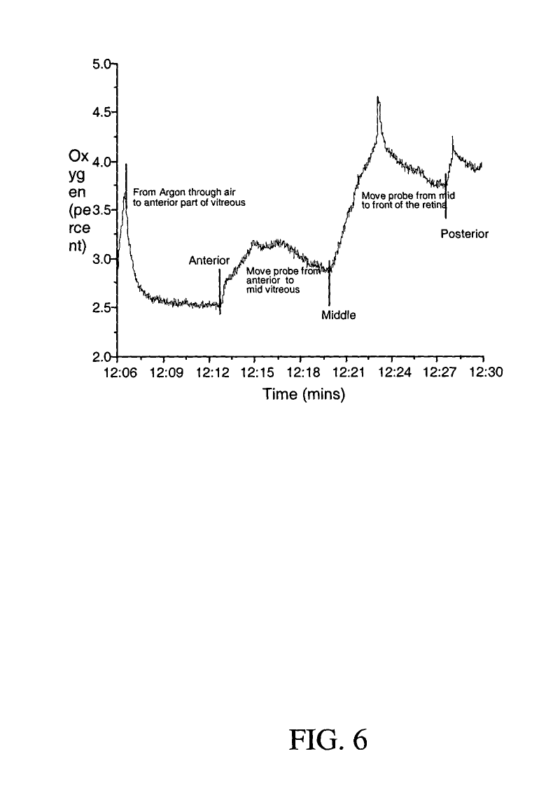

FIG. 6 similarly illustrates low oxygen levels in the vitreous of a rabbit eye.

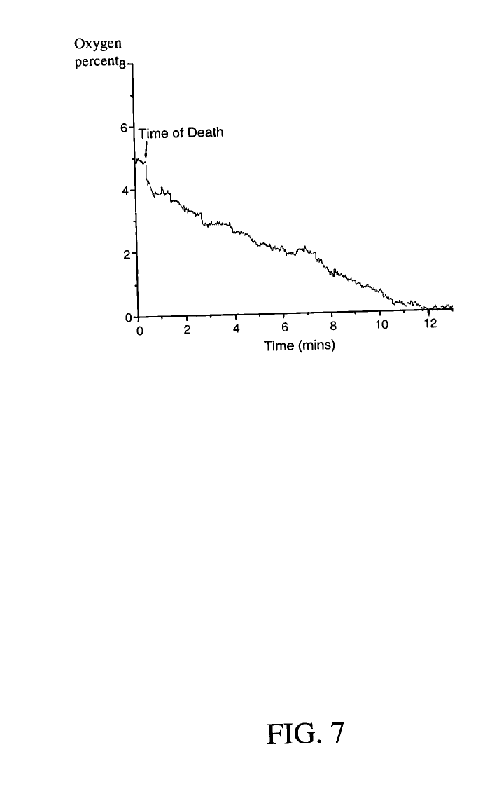

FIG. 7 depicts oxygen decrease in a rabbit vitreous over time (after death), and indicates a rate of decrease that cannot be explained by diffusion alone. Hence, the data of FIG. 7 provide evidence of chemical processes taking place in the vitreous that consume oxygen.

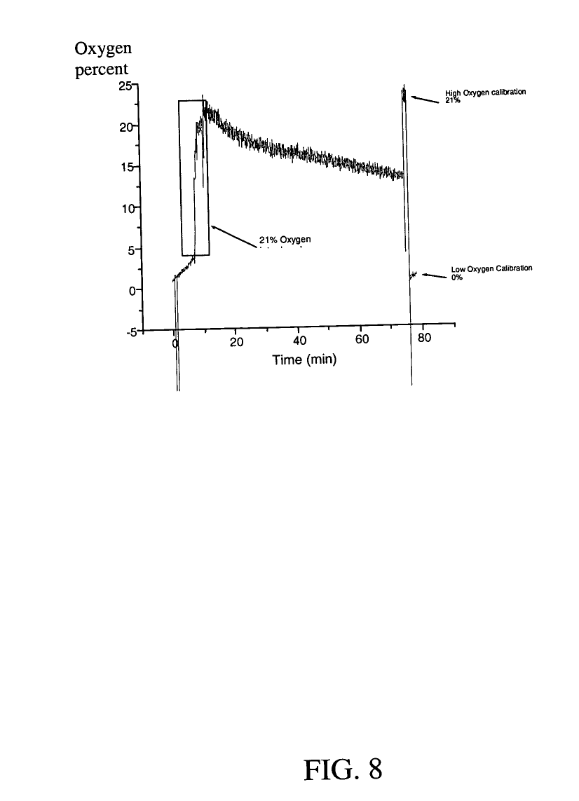

FIG. 8 shows decrease in oxygen in an isolated rabbit vitreous over time, after bubbling with 21% oxygen. The decrease rate shown in FIG. 8 indicates that chemical processes in the vitreous are consuming oxygen. Since FIG. 8 involves an isolated vitreous, the data of FIG. 8 eliminate the possibility that retinal chemical reactions cause the high rate of oxygen reduction. The data of FIG. 8, therefore, provide strong evidence of chemical reactions in the vitreous that consume oxygen, and, therefore, contribute to the low oxygen concentration of the vitreous.

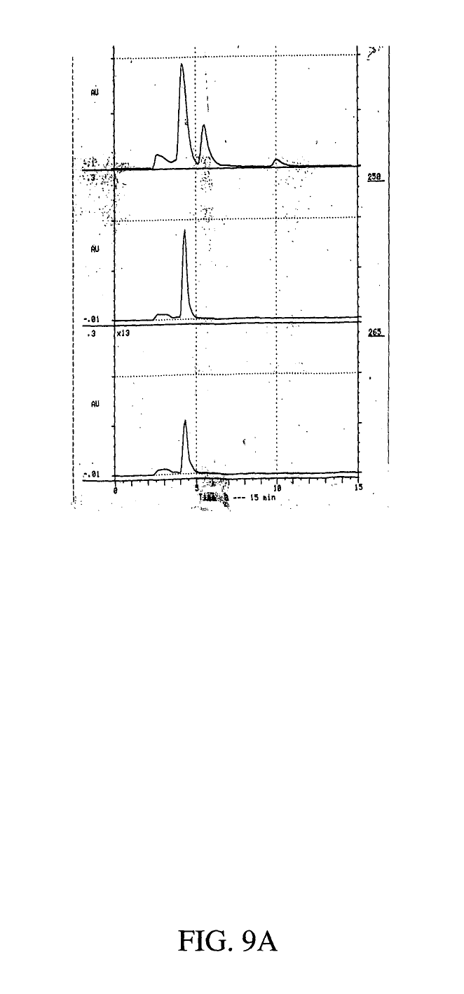

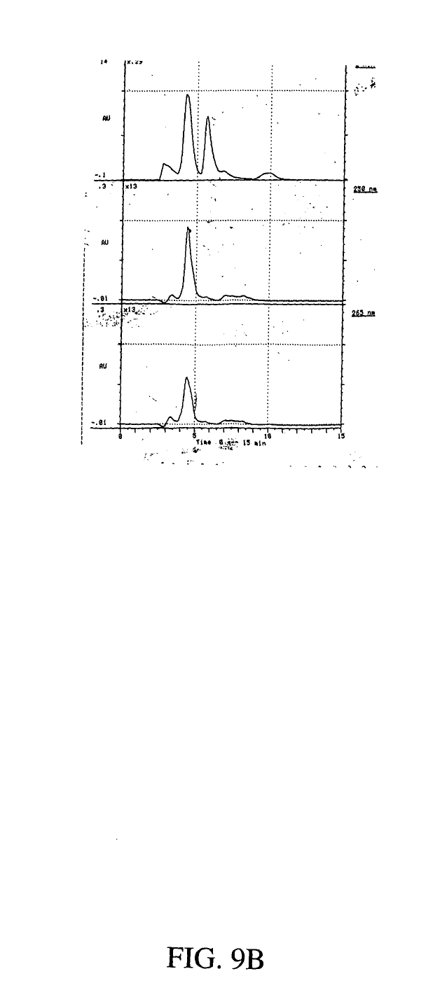

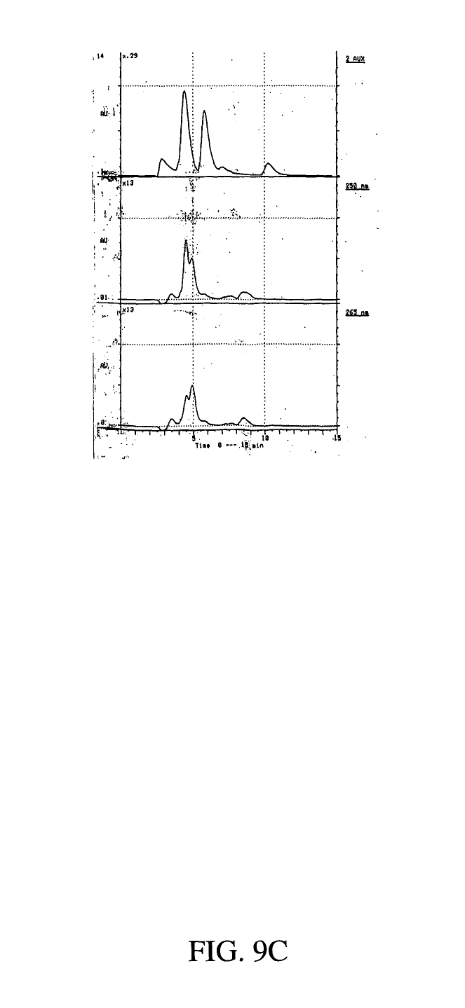

FIG. 9 depicts actual chromatogram traces of a rabbit vitreous. Peak 902 of FIG. 9A clearly indicates the consumption of ascorbic acid in the vitreous, leading to the formation of numerous chemical products. The data of FIG. 9 provide strong evidence that chemical reactions in the vitreous, involving ascorbic acid, contribute to oxygen consumption in the vitreous.

Since higher-than-normal levels of oxygen in the vitreous (which is normally almost anaerobic) are believed to be a cause of cataract development, it is expected that measures to reduce oxygen levels in the vitreous can protect against cataract development in a subject. For example, in one embodiment of the present invention, measures are taken to reduce abnormally-high vitreous oxygen levels to normal vitreous oxygen levels. As used herein, oxygen levels that are abnormally high (i.e., abnormally higher than normal) range from about 5% to about 21%. A “normal” vitreous oxygen level refers to a concentration from about 1% to about 5%. Subjects in a high risk of cataract development are likely to benefit from the methods and compositions of the present invention, particularly older subjects and individuals who have undergone vitrectomies or hyperbaric oxygen eye treatments. However, embodiments of the invention are also applicable to subjects who do not have (or are not known to have) a high risk of developing cataracts. In addition, in some embodiments, the methods of the invention are applied to advantage in any type of animal having eyes.

In accordance with some embodiments, the present invention provides a method for protecting against cataract development by decreasing vitreous oxygen concentration in subjects to less than about 5%. Preferably, the vitreous oxygen is decreased to a concentration from about 0% to about 3%, and, most preferably, to a concentration from about 0% to about 2%. The vitreous oxygen concentration can be decreased, for example, by use of a vitreous replacement solution having a low oxygen concentration.

In accordance with the present invention, ascorbic acid plays a significant role in oxygen-consuming chemical reactions that occur as part of the metabolic processes taking place in the vitreous. In fact, increased levels of ascorbic acid in the vitreous can cause increased metabolism and consumption of oxygen facilitated by enzymes in the vitreous, thereby lowering vitreous oxygen concentration and protecting against cataract development.

While ingestion of ascorbic acid (vitamin C) can increase ascorbic acid levels in the vitreous, such ingestion will only increase vitreous ascorbic acid levels to a certain maximum, beyond which additional ascorbic acid ingestion will not increase ascorbic acid levels in the vitreous. Accordingly, in one embodiment of the present invention, optical solutions containing ascorbic acid are administered to eyes of subjects (e.g., by eye drops), to provide a dosage of ascorbic acid effective to prevent cataract development. Effective dosages of ascorbic acid in the eye drops can include any dosage up to about 10 mM (millimolar). Preferably, the effective dosage of ascorbic acid ranges from about 0.5 mM to 5 mM, and, most preferably, ranges from about 1 mM to about 3 mM. A particularly preferred effective dosage of ascorbic acid is 2 mM. Similar concentration ranges apply to ascorbic acid dosages in vitreous replacement solutions.

Some embodiments of the present invention relate to vitrectomies. Typically, in vitrectomies, the natural vitreous is removed and replaced by a vitreous replacement substance, such as an ophthalmic irrigating solution. Examples of ophthalmic irrigating solutions include, without limitation, Balanced Salt Solution (BSS® or BSS Plus®), both of which are commercially available from Alcon Laboratories, Inc. (Fort Worth, Tex.). Nuclear cataracts typically develop within a year after vitrectomies, and the risk is especially great in older vitrectomy patients. Ophthalmic irrigating solutions, such as BSS® and BSS Plus®, are generally air-saturated. Hence, after vitrectomies, the vitreous contains much higher levels of oxygen—obtained from the high oxygen content of the irrigating solution. It is believed that normal (nearly anaerobic) vitreal oxygen levels are thought eventually to be re-established as the aqueous replaces the irrigating solution.

It is observed, however, that, following vitrectomies, and while normal vitreal oxygen levels have yet to be re-established, oxygen diffusion from the vitreous to the lens is greater-than-normal, leading to greater-than-normal oxygen concentrations in the lens (which is normally nearly anaerobic). Given that high levels of oxygen in the vitreous and lens are believed to lead to cataract development, it is expected that the high levels of oxygen in air-saturated vitreous replacement solutions, such as BSS® and BSS Plus®, will lead to cataract development.

For these reasons, another embodiment of the invention provides a vitreous replacement solution having a low oxygen concentration, for use in a vitreous replacement to protect against cataract development. As used herein, a vitreous replacement solution having a “low oxygen concentration” is a vitreous replacement solution with an oxygen concentration of about 2% or less. In some embodiments, the vitreous replacement solution has an oxygen concentration from about 0% (e.g., is essentially oxygen-free) to about 5%, and is preferably essentially oxygen-free. As discussed below, an essentially-oxygen-free solution is preferable in vitreous replacement solutions in which GSH (reduced glutathione) or ascorbic acid, or a combination thereof, is used, due to the stability of ascorbic acid and/or GSH in such a solution.

In some embodiments, vitreous replacement solutions having lower oxygen concentrations than air-saturated vitreous replacement solutions are used in vitrectomies or during vitreous replacements. For example, in some embodiments, nitrogen, or another essentially-oxygen-free inert gas, such as a noble gas, is bubbled or otherwise introduced into an initial (e.g., air-saturated) vitreous replacement solution, such as BSS Plus®, prior to its use during a vitrectomy, to remove some or essentially all of the oxygen in the solution. By way of example, nitrogen gas may be bubbled through BSS Plus® solution for about 5-20 min, or for about 10 min, immediately prior to its use during a vitrectomy. As another example, reduced oxygen may be achieved by subjecting the initial solution to a vacuum, at various levels of reduced pressure, typically for about 10-15 min, depending on the level of the vacuum applied, with or without bubbling gases prior to or after application of the vacuum to the solution. Many other embodiments involving reduced oxygen vitreous replacement solutions, and ways to reduce oxygen in the initial (e.g., air-saturated) solutions, will be recognized by one skilled in the art. In some embodiments, essentially-oxygen-free vitreous replacement solutions are utilized. Additionally, in certain embodiments, the lower-oxygen vitreous replacement solution can be a gel or have some other form.

Lower-oxygen vitreous replacement solutions are believed to have an additional advantage to those already discussed. Solutions such as BSS Plus®, in its initial (air-saturated) form, contain oxidized glutathione, or GSSG. Through the action of enzymes in the eye, GSSG is converted into glutathione reductase, or GSH (reduced glutathione), which is an anti-oxidant believed to protect eye structures. It is generally not practical to introduce GSH directly into the eye (e.g., by adding it to BSS Plus® and using the BSS Plus® as a vitreous replacement), because, in air-saturated BSS Plus®, the GSH is quickly oxidized to GSSG. However, in lower-oxygen or essentially-oxygen-free solutions, such as nitrogen-saturated BSS Plus®, as utilized in some embodiments of the invention, GSH is not quickly reduced into GSSG; instead, it will remain as GSH in the solution, at least for a significant period of time. Therefore, GSH may be added to lower-oxygen or essentially-oxygen-free vitreous replacement solutions according to some embodiments of the invention, so as to introduce GSH directly into the eye during a vitrectomy. Effective GSH concentrations (e.g., from about 0.01 mM to about 10 mM; preferably from about 0.1 mM to about 2 mM; and most preferably about 1 mM) are useful additions to the low-oxygen or essentially-oxygen-free solutions provided herein. By adding GSH directly into the eye via addition of lower-oxygen or oxygen-free vitreous replacement solutions, the presence and quantity of GSH in the eye is not dependent upon, or limited by, the action of ocular enzymes in reducing GSSG to GSH.

In some embodiments of the present invention, ascorbic acid is included in the vitreous replacement solution. The benefits of ascorbic acid in protecting against cataract development have been discussed above. In other embodiments, various methods and compositions of the invention are utilized to treat cataracts, such as by reducing the severity of cataracts or eliminating cataracts.

The present invention is described in the following Examples, which are set forth to aid in the understanding of the invention, and should not be construed to limit in any way the scope of the invention as defined in the claims which follow thereafter.

### EXAMPLES

### Example 1

### Preliminary Experiments and Introduction to Oxygen Tension Study

Presented below is a series of experiments for determining oxygen partial pressure (pO₂) in various ocular structures using either an optode or a micro Clark oxygen electrode. An optode (Oceanoptics Corp., Dunedin, Fla.) measures oxygen via photophysical processes in which a signal is inversely proportional to oxygen tension. As such, it is most sensitive at low oxygen tensions, similar to those found in the lens. In addition, an optode is specifically designed for viscous media, which also makes it appropriate for lenticular studies. However, it is disadvantageous in that it is sensitive to light. To overcome this disadvantage, the optode is normally covered with a silicone outer coating. As a result, it takes some time for the optode to reach equilibrium. This characteristic varies, depending upon the specific optode employed, as observed by the inventor who has examined numerous optodes. Therefore, the equilibrium times set forth below vary from one experiment to another, but are quite consistent for each repetitive reading for an individual optode. The lowest level reached gives an accurate determination of the actual pO₂ in a tissue or solution.

Several studies have demonstrated that an oxygen gradient exists from the retina to the posterior of the lens (Alder et al., Vitreal oxygen tension gradients in the isolated perfused cat eye. Curr. Eye Res., 5:249; Sakaue et al., Measurement of vitreous oxygen tension in human eyes. Jpn. J. Ophthalmol., 33:199-203, 1989). Much of this gradient can be explained on the basis of consumption of oxygen in the retina, but evidence is presented herein that there are chemical processes in the vitreous that consume oxygen. These processes may involve the reaction of two oxygen molecules with ascorbic acid, resulting in the formation of hydrogen peroxide. The subsequent degradation of hydrogen peroxide by various enzymes in the vitreous leads to the formation of one molecule of oxygen. Therefore, each cycle leads to the loss of one oxygen molecule, similar to the process suggested by Eaton (Is the Lens canned? Free Radical Biol. and Med., 11:207-13, 1991). It is important to note that the only antioxidant with which cataracts are negatively correlated is ascorbic acid (Jacques et al., Long-term nutrient intake and early age-related nuclear lens opacities. Arch. Ophthalmol., 119:1009-19, 2001), and that there is decreased ascorbic acid in cataracts (Tessier et al., Decrease in vitamin C concentration in human lenses during cataract progression. Internat. J. Vit. Nutr. Res., 68:309-15, 1998). This latter point suggests that oxygen is increased in the lens during cataract formation.

Both in vitro and in vivo methods are presented herein to measure oxygen tension in the lens using an optode in conjunction with an oxygen electrode. This technique serves to determine relative changes in oxygen during the course of an experiment. However, there is one fundamental problem in ascertaining absolute oxygen concentrations using these probes: pO₂ is measured, rather than concentration. In both techniques, oxygen has to pass through a membrane to get to the detector. The actual concentration in solution is dictated by Henry's Law, which states that the ratio between pO₂ and dissolved oxygen (Henry's constant) is invariant with respect to changes in pressure. This assumes that the buffer is approximately the same for the standard (where the electrode is standardized) versus experiment. However, it is well known that high salt or compounds like amino acids or glycerol (Rischbieter and Schumpe, Gas solubilities in aqueous solutions of organic substances. J. Chem. Eng. Data, 41:809-12, 1996; Schumpe, A., The estimation of gas solubilities in salt solutions. Chemical Engineering Sci., 48:153-58, 1993) can drastically increase Henry's constant versus water, thereby increasing error using an electrode or optode.

FIG. 1A depicts the change of Henry's constant with increasing glycerol in water solutions (Rischbieter and Schumpe, Gas solubilities in aqueous solutions of organic substances. J. Chem. Eng. Data, 41:809-12, 1996). Since there is an increase in Henry's constant with increasing glycerol, the measured pO₂ will give over-estimations of the actual oxygen concentration. FIG. 1B depicts the actual concentration of oxygen measured by the Winkler method (concentration curve) with increasing glycerol/water mixtures. Also included is the inventor's measurement of oxygen tension using an oxygen electrode. Both experiments were performed in air-saturated solutions. The X axes in these figures are not the same; nevertheless, they clearly show that there can be a large error in the determination of oxygen tension when solution content is varied. It is essential, therefore, to determine Henry's constant for the vitreous, and especially the lens, in order accurately to determine the actual concentration of oxygen in those compartments.

General Methodology:

Sources of the Human Lenses

Human lenses may be obtained from the New York Eye Bank (New York, N.Y.). Approximately five per month of various ages may be obtained. They may be stored at −70° C. until used.

Rabbits Used

Hare Marland rabbits may be used in these studies. The animals should be handled in accordance with institutional guidelines for animal research, and with the ARVO statement for the use of animals in ophthalmic and vision research.

Data Analysis

For data analysis, curve fitting, and statistics, Origin 6.0 data analysis and graphing software available from Microcal LLC (Northampton, Mass.) may be used. This is a very sophisticated package, and contains all of the standard statistical analyses and numerous curve-fitting routines.

EPR Oximetry

For all oximetry measurements, the TPX capillary is used. This capillary (˜0.6 mm ID) is made of a pentene polymer (called TPX) which is permeable to oxygen, nitrogen, and other gases, but is substantially impermeable to water (Subczynski et al., Oxygen permeability of phosphatidylcholine-cholesterol membranes. Proc. Natl. Acad. Sci., USA. 86:4474-78, 1989). Samples placed inside the capillary can be easily equilibrated with the gas blowing outside the capillary, whether it is nitrogen, air, or an air/nitrogen mixture. This gas (mixture) is also used for temperature control, so that the sample can be equilibrated, at a certain temperature, with known oxygen partial pressure.

Tissue Culture

A number of investigators have described growth medium suitable for maintenance of lens transparency, metabolism, and physiology (Kleiman et al., Hydrogen peroxide-induced DNA damage in bovine lens epithelial cells. Mutation Res., 240:35-45, 1990; Kleiman et al., Ultraviolet light induced DNA damage and repair in bovine lens epithelial cells. Curr. Eye Res., 9:1185-95, 1990; Kleiman et al., In Duane's Clinical Ophthalmology, W. Tasman and E. A. Jaeger, eds. (Philadelphia: J.P. Lippincott & Co., 1994), vol. 1, ch. 15, pp. 1-39; Spector et al., Repair of H₂O₂ induced DNA damage in bovine lens epithelial cell cultures. Exp. Eye Res., 49:685-98, 1989; Spector et al., A brief photochemically induced oxidative insult causes irreversible lens damage and cataract II. Mechanism of Action. Exp. Eye Res., 60:483-92, 1995a; Spector et al., Development and characterization of an H₂O₂-resistant immortal lens epithelial cell line. Invest. Ophthalmol. Vis. Sci., 41:832-43, 2000). Some of the more experimentally-useful systems are cell-culture-medium-based, often Hepes-buffered, contain serum, and may be supplemented with additional growth-promoting components. In the studies presented herein, a variety of such media may be tested in a rabbit lens culture system, including artificial aqueous medium (Richer and Rose, Water soluble antioxidants in mammalian aqueous humor: interaction with UV B and hydrogen peroxide. Vision Res., 38:2881-888, 1998), and many of the physiological parameters discussed below may be utilized to ascertain whether the lens is physiologically well maintained. The ultimate goal of such a buffered medium is to reproduce, as closely as possible, the physiological and physiochemical properties of rabbit aqueous. Rabbit vitreous flow rates are in the range of ˜3.0 μl/min, and this rate may be used to perfuse the lenses in the chamber.

Epithelial Cell Damage

Reactive oxygen species (ROS), primarily H₂O₂, superoxide, and the hydroxyl radical, are formed by visible light irradiation of organ-cultured lenses in a medium containing riboflavin (Spector et al., A brief photochemically induced oxidative insult causes irreversible lens damage and cataract II. Mechanism of Action. Exp. Eye Res., 60:483-92, 1995a). In a 4% oxygen environment, similar to the oxygen tension at the anterior surface of the lens, the amount of H₂O₂ and ROS can be adjusted by varying the concentration of riboflavin. By varying light exposure, it is possible to create conditions of oxidative stress that lead to reversible or irreversible changes in the lens epithelium, eventually leading to lens opacification (Spector et al., A brief photochemically induced oxidative insult causes irreversible lens damage and cataract II. Mechanism of Action. Exp. Eye Res., 60:483-92, 1995a). Thus, titratable insult to the epithelium can be utilized as an experimental tool with which to damage or kill the anterior epithelium of organ-cultured rabbit lenses, and facilitate measurements of lens oxygen diffusion and concentration.

Quantification of Damage

In order to quantify and assess changes to the epithelium as a consequence of photooxidative insult, three experimental approaches may be used. The first, measurement of cell viability by Trypan blue exclusion and/or live/dead staining assays, can establish the geographical pattern and time course of epithelial cell death following insult, and has been previously utilized to examine oxidative stress in organ-cultured rat lenses under a variety of conditions (Spector et al., A brief photochemically induced oxidative insult causes irreversible lens damage and cataract II. Mechanism of Action. Exp. Eye Res., 60:483-92, 1995a). The second, measurement of active transport through the lens epithelial cell membrane, utilizing choline and rubidium uptake, is a more sensitive indicator of early changes in the epithelium. Significant decreases in these values can be demonstrated within the first hour following insult (Spector et al., A brief photochemically induced oxidative insult causes irreversible lens damage and cataract I. transparency and epithelial cell layer. Exp. Eye Res., 60:471-81, 1995). Lastly, measurement of glyceraldehyde 3-phosphate dehydrogenase (GPD), a key enzyme in carbohydrate metabolism, provides a measure of changes in the lens's ability to produce energy (Spector et al., Development and characterization of an H₂O₂-resistant immortal lens epithelial cell line. Invest. Ophthalmol. Vis. Sci., 41:832-43, 2000).

Briefly, dye exclusion studies require removal, fixation, and preparation of a flat mount of the lens epithelium, which is then examined by light or fluorescence microscopy; positively-stained cells are recorded. It is possible to measure quantitatively the degree of viability of the epithelial monolayer, by simply comparing the numbers of dead and viable cells.

¹⁴C-choline- and ⁸⁶Rb-uptake studies involve incubation of lenses with the radioactive compound, careful washing, removal of the epithelium, homogenization, and scintillation counting. As Rb-uptake measurements mimic Na/K-ATPase-catalyzed potassium-ion transport, confirmation of this effect can be obtained by using ouabain inhibition of the ATPase (added to the medium prior to addition of rubidium).

GPD measurements are based on the enzyme activity protocol first described by Beyers (Glyceraldehyde-3-phosphate dehydrogenase from yeast. Methods Enzymol., 89:326-35, 1982), and modified for use in the lens (Spector et al., The prevention of cataract caused by oxidative stress in cultured rat lenses. I. H₂O₂ and photochemically induced cataract. Curr. Eye Res., 12:13-179, 1998). They involve homogenizing the tissue in bicine/Triton-X buffer, and assaying the supernatant, after addition of substrate and co-factors, at 340 nm.

Animal Studies

Large eyes are needed to make the procedure feasible, and to allow evaluation post-surgery. Rabbits are readily available, and are commonly used for projects of this type. At least 50 animals are needed to determine: (1) the effect of vitreous surgery on dynamics of oxygen tension in the eye at various points post-surgery; (2) changes of light transmission due to cataract formation post surgery; and (3) safety and usefulness of the procedure prior to measurements in humans. Aqueous fluids with different oxygen tensions are used, to study the effect on intraocular oxygen tension. The partner eye of the animal is used as a control at the end of the study. Approximately 5 animals per group are needed for significant results. A fully certified animal facility, with all of the attendant veterinary care and other support staff, is utilized.

Ketamine (35 mg/kg) and xylazine (5 mg/kg) are used as anesthetic agents (IM) when surgery is performed. Anesthesia is given and maintained by IM-administered ketamine and xylazine. In order to provide adequate levels of sedation, a standard dose/kg table is utilized. Adequate sedation can be defined as a state where the rabbit is unconscious and immobilized, and is monitored by observing response to pain as indicated by eye or body movement. Additional anesthetic is administered, if necessary, to maintain unconsciousness only while carefully observing the animals to ensure no development of respiratory depression. One member of a surgical team may monitor anesthesia.

Surgical procedures, including measurements of light and oxygen, take between 60 and 120 min. All manipulations are visualized using a self-adhering contact lens and an operating microscope. Euthanasia is performed under general anesthesia at the end of the last surgical procedure, using IP pentobarbital (100 mg/kg).

After dilation of the pupil, the nictitating membrane of the eye is excised. Four to six continuous spots of cryotherapy are applied 6 mm posterior to the limbus, just inferior to the medial rectus muscle and the long posterior ciliary artery. Two weeks later, the rabbit is placed under general anesthesia again, and the pupil is dilated. A sclerotomy is placed 1 mm posterior to the limbus in the area of previous cryopexie. Oxygen levels are measured before vitrectomy, directly after vitrectomy, and during a second procedure under general anesthesia at 1-4 weeks following the vitreous surgery. For the oxygen measurement, a Clark-style oxygen microelectrode is employed through one of the scleral incisions; it measures oxygen tension in different locations within the eye: towards the retina, half way to the posterior of the lens, close behind the lens, close to the limbal area, and on the other side.

Prior to vitrectomy, 2 additional sclerotomies are placed 1 mm posterior to the limbus, in the area of previous cryopexie. Vitrectomy is performed using a vitreous cutter probe (Ocutome® vitreoretinal equipment, available from Alcon Laboratories, Inc., Fort Worth, Tex.), a light pipe, and a balanced salt solution, according to standard protocol. After the scleral incision and the first measurements, an infusion needle (23 g butterfly) with balanced salt solution is inserted to provide infusion fluid 3 mm from the limbus. The microvitrectomy cutting instrument is inserted through sclerotomy, for cutting and removal of part of the vitreous. After vitrectomy, an aqueous solution of known oxygen level is installed into the eye. Directly after installation of the solution, oxygen measurements are repeated using a microelectrode. All of the instruments are then removed, and the small scleral incisions are closed with 9-0 nylon and topical bacitracin/gentamicin. Atropine or cyclogel is then applied to the eye.

### Example 2

### Oxygen Tension Experiments

Studies may be performed to ascertain whether the optode of the present invention is useful in accurately assessing oxygen tension in various compartments of the eye. FIG. 2 depicts the results for a cow eye, approximately 4 h after slaughter. The aqueous, vitreous, and lens have 5%, 1%, and 1%-2% oxygen, respectively. The concentrations of the aqueous are in reasonable agreement with the literature (Kwan et al., In vivo measurements of oxygen tension in the cornea, aqueous humor, and anterior lens of the open eye. Inves. Ophthalmol. Vis. Sci., 11:108-14, 1972), but the concentrations of the vitreous (which are normally in the range of 3-5%, or 20-40 mmHg) are not. This, at first, was puzzling; however, it is most likely due to the very active nature of the vitreous, as described below.

The main problem in measuring oxygen concentration in the lens is that the probe, when air-saturated, takes a great deal of time to come to equilibrium. This characteristic may be exploited, since the rate of equilibration depends on the viscosity of the solution.

Diffusion of Oxygen

In order to determine the pO₂ of a solution, oxygen (as a gas) must enter or leave the optode matrix until equilibrium is reached with the solution. If the optode is air-saturated, and is placed in a de-aerated buffer, oxygen will leave the matrix rapidly, for a relatively fast equilibrium. However, as the solution becomes more viscous, the rate at which equilibrium is reached slows in a direct relationship with the viscosity of the solution. This is explained by the Stokes-Einstein equation, which states that diffusion of a gas in a solution is inversely proportional to the viscosity of that solution. FIG. 3A depicts the traces for the loss of oxygen from an optode with solutions of increasing viscosity. The amount of oxygen detected by the optode decreases exponentially, until it reaches the actual oxygen concentration of the solution. The amount of time to reach this equilibrium increases for increasing viscosity.

FIG. 3A depicts typical decays of the optode signal when the optode is taken from air and placed into a solution saturated with argon. As the viscosity of the glycerol/water solution increases, the decay time also increases. FIG. 3B shows the calculated exponential decay versus viscosity. This was determined by fitting the curves to first-order exponential decays using Origin 6.0 data analysis and graphing software available from Microcal LLC (Northampton, Mass.). Clearly, the fits are linear with viscosity.

Diffusion of Oxygen in the Lens

The investigation of diffusion of oxygen into the lens was a significant aim herein. As depicted in FIG. 4, a 300-μm diameter optode, with the tip covered with silicone, was inserted into the center of a calf lens. The lens was then incubated in air-saturated phosphate buffer, at 37° C. The initial part of the graph in FIG. 4 consists of oxygen diffusing out of the probe, and reaching the basal level of the lens. Thereafter, oxygen diffuses into the lens. The final part of the curve (at 40 h) shows a test for the stability of the probe. The probe was clearly stable over the time course of the experiments.

When the rates of decay in cow and pig lenses were compared to the decay in glycerol/water mixtures, it was found that the viscosity of both pig and cow lenses were approximately 10-12 cp at 40° C., and 15-16 cp at 27° C. Since the Stokes-Einstein equation shows an inverse relationship between viscosity and diffusion, these results suggest that oxygen will diffuse some 1-2 orders of magnitude slower in a lens, compared to water (1 cp). This is a new method to determine the viscosity and the diffusion of oxygen in viscous solutions. It should be noted that a recent paper (Dierks et al., Protein size resolution in human eye lenses by dynamic light scattering after in vivo measurements. Graefe's Arch. Clin. Exp. Ophthalmol., 236:18-23, 1998) discussing light scattering in intact lenses described 2 cp as an estimation of the viscosity of the lens. This was based on the viscosity of concentrated protein solutions, and is clearly an underestimation.

In testing various lenses at various times after slaughter, it was noted that basal oxygen levels could vary from approximately 1% to as high as 5-10%, depending on the length of time after slaughter. It seemed clear that oxygen was diffusing into the lens. In order to determine the time for this process, a cow lens (only 2 h after slaughter) was incubated in air-saturated buffer at 37° C. (below). There was an initial decrease to approximately 1% oxygen, as oxygen diffused out of the optode; then, over a period of 30-40 h, there was an increase in oxygen from 1% to 5% in the center of the cow lens. By comparing the volume of the cow lens to the human lens, it was estimated that this process would take approximately 4 times less to reach 5% oxygen in human lenses.

Live Rabbit Lens

To determine the actual concentration of oxygen in a mammalian lens, experiments on live rabbits were performed. It is clear that the time that the optode takes to come to equilibrium limits its usefulness for in vivo experiments in the lens. Therefore, a tiny (270-μm tip) Clark oxygen electrode (Diamond General Corp., Ann Arbor, Mich.) was also used. This electrode is small enough that the consumption of oxygen is minimal. Thus, the various attributes of the optode and oxygen electrode make them complimentary, as discussed herein.

FIG. 5 depicts the concentration of oxygen in the lens. The Y-axis is not absolute, but relative. The last point, at approximately 8.6 mm, is outside the lens, and in the vitreous. To produce the data depicted in the figure, the oxygen electrode was inserted into the center of a live rabbit lens, and slowly pushed through with a micromanipulator. Clearly, oxygen is asymmetrically present in the lens. The anterior is at a much higher tension than the posterior, which, in turn, is very close to the tension found in the vitreous. Oxygen tension was very high near the epithelial layer, but fell off dramatically in the inner cortex, with a minimum in the center (with lens axial width approximately 8.5 mm). Similar results were obtained for a young monkey lens (within 2 h of death).

This experiment supports the contention that part of the development of the lens is controlled by oxygen starvation in the inner cortex. It does not prove, but correlates well with, final fiber formation. In addition, the results obtained from this experiment may be used as an assay, to monitor precisely changes in oxygen tension as the environment surrounding the lens is manipulated for in vitro and in vivo experiments.

Live Rabbit Vitreous Humor

As stated above, the oxygen tension of vitreous obtained from cadaver eyes was 1%, which is much less than that found in live animals. To compare the results obtained herein with the results of other experiments, the optode was used on the vitreous of a live rabbit. In particular, to produce the data depicted in FIG. 6, the optode was inserted into the anterior vitreous of an anesthetized rabbit eye. It was then slowly moved toward the posterior, and allowed to stabilize at two points. Even though this experiment was performed by hand, a micromanipulator may be used.

Although there was some noise (since the probe was inserted by hand), results showed a gradient from anterior to posterior, in the range of 2.5-4.0% (20 mmHg-30 mmHg). This reasonably agrees with published results (Alder et al., Vitreal oxygen tension gradients in the isolated perfused cat eye. Curr. Eye Res., 5:249; Sakaue et al., Measurement of vitreous oxygen tension in human eyes. Jpn. J. Ophthalmol., 33:199-203, 1989).

Euthanized Rabbit

To determine what occurs in the rabbit eye after the blood supply is cut off, a rabbit was euthanized and the oxygen tension in the center of the vitreous was monitored with the optode. FIG. 7 shows, surprisingly, that oxygen tension in the vitreous went to zero in a remarkable 10-12 min. Based on fluorescein diffusion, it has been estimated that a molecule the size of oxygen should take ½ h to clear 50% of its concentration. The process detected above is considerably faster than that which can be explained by simple diffusion out of the vitreous, and suggests that other processes are involved.

Isolated Vitreous

A major factor affecting the above-described loss of oxygen may be the robust metabolism of the retina, as the retina would continue to metabolize for some time after death. Another possibility is that there are other factors in the vitreous to dispose of oxygen, including chemical reactions. To test this, vitreous humor was isolated from newly-euthanized rabbit eyes, and was bubbled with 21% oxygen, as show in FIG. 8. The percent concentration of oxygen was then followed over time using the optode.

According to the results depicted in FIG. 8, there was a loss of oxygen of almost 10% over 40 min. Since this sample no longer contained the retina, such decreases cannot be due to retina metabolism. However, there was a clear decrease in oxygen tension, suggesting that a chemical process had taken place. Similar results were obtained with a cow vitreous (within 2-4 h of slaughter). However, eyes stored much longer did not give positive results, and vitreous exposed to air quickly deteriorated in terms of its ability to dispose of oxygen. Therefore, this appears to be a sensitive and dynamic process. The last part of the graph in FIG. 8 shows a test of the accuracy of the optode.

HPLC Studies

In an attempt to understand the mechanism(s) of oxygen disposal, reactions were followed by HPLC, using photodiode array and electroanalytical detection. This method can simultaneously detect ascorbic acid, GSH, Tyr, and other reducible constituents of the vitreous. FIG. 9 sets forth chromatogram traces of vitreous (buffer added in equal amounts and centrifuged) from an anaesthetized live rabbit (FIG. 9A), from a rabbit 10 min after sacrifice (FIG. 9B), and that was isolated, bubbled with oxygen, and allowed to stand for 10 min (FIG. 9C). In each figure, the top chromatogram is from an electroanalytical detector set at 20 μA, and the other two are from a photodiode array detector set at 250 and 265 nm. The column was a reverse phase C18, with a buffer consisting of potassium dihydrophosphate adjusted to pH 3.0 with ortho-phosphoric acid.

The second peak (902) of FIG. 9A is ascorbic acid. There is a clear broadening of the peak in FIG. 9B, and the clear appearance of a new peak in FIG. 9C. In addition, while the chromatogram of FIG. 9A is clean, there appear numerous other components in FIGS. 9B and 9C. Evidently, ascorbic acid is being consumed in these reactions, resulting in the formation of numerous products. It must be emphasized that the detected reactions were readily observed after only 10 min of blood-supply cutoff. This is a very dynamic system, and strongly suggests, in part, that an oxygen gradient exists from the retina to the posterior of the lens due to chemical reactions that involve ascorbic acid.

### Example 3

### Experimental Design and Methodology

Presented herein is a significant, yet very simple, hypothesis: that the apparent low levels of oxygen in the lens are essential for its development and the maintenance of its transparency. The simplicity, however, ends there. Very little is known concerning the oxygen levels in the lens, the diffusion of oxygen within various portions of the lens, and changes in oxygen tension relating to age and/or environment. This is due to the inherent difficulties in measuring oxygen in a viscous tissue like the lens. One approach to these issues, provided herein, is to investigate several physical parameters of oxygen (tension, diffusion, etc.) on the macroscopic level and in the lens as a whole lens; to investigate, at the same time, detailed oxygen permeability in various microscopic lenticular structures; and, finally, to extend the results from the macroscopic experiments to a rabbit model.

As discussed herein, one aim of the present Examples is to use an optical oxygen probe (optode) and oxygen electrode to determine the concentration and macroscopic diffusion of oxygen in the intact mammalian lens. Experiments may be performed to determine the effect of environmental changes around the lens on oxygen concentration within the lens. Studies may also be performed in a chamber that isolates the anterior from the posterior lens. This may be used to determine the consequences of epithelial cell dysfunction on the oxygen concentration of the lens, and may serve as a model for senile nuclear cataracts.

Principle of Luminescence Detection of Oxygen

The optode is a fiber-optic probe coated with a ruthenium complex at the distal end of it. This material is entrapped in a hydrophobic matrix, and is protected from water. The light source emits a wavelength of 475 nm, and excites the complex. The excited complex fluoresces, and emits a 600-nm light. When the excited complex encounters an oxygen molecule, the energy is transferred to the oxygen molecule in a non-radiative manner. This transfer of energy decreases the fluorescence signal, and is dependent on the concentration of oxygen molecules.

The Stern-Volmer equation relates the fluorescence to the concentration of oxygen quantitatively:

(I_{O}+I)=1+kC

where I_{O} is defined as the fluorescence at zero concentration of oxygen; I is the intensity of concentration C of oxygen; and k is the Stern-Volmer constant. The fiber-optic probe is as small as 300 μm, and the system is completely computer-controlled for real-time data acquisition. The probe is first standardized with solutions of known percentages of oxygen, and then oxygen in unknown samples is measured.

Obtaining the absolute oxygen concentration of a viscous tissue like the lens presents many problems. As presented herein, two oxygen probes have been used: an optode, as described above, and an oxygen electrode. These are based on two different physical principles, but are essentially complimentary. The optode can be used for media of widely-varying viscosity; however, it is very slow in response, and response time increases markedly with increasing viscosity. Therefore, it has limited utility for animal experiments. Nevertheless, the inventor believes that the optode gives accurate pO2 values in viscous media. This is based on numerous experiments using various solutions of glycerol/water and sucrose/water mixtures, where the Henry constants are known (Rischbieter et al., Gas solubilities in aqueous solutions of organic substances. J. Chem. Eng. Data, 41:809-12, 1996). Conversely, the oxygen electrode responds rapidly, and is ideal for dilute solutions. However, it can have large baseline shifts when inserted into the lens. Thus, the oxygen electrode may be used to probe relative oxygen tension, through regions of the lens, using a micromanipulator, while the optode may be used to obtain a single, accurate number in the same experiment.

Henry's Constant

The oxygen probes used in the present studies were designed to measure pO₂, not oxygen concentration per se. They are very good for showing changes in oxygen tension over time, but do not give an absolute value of oxygen concentration. When the probes are standardized, water or dilute protein solution is usually used. In this situation, Henry's constant is known; therefore, actual oxygen concentration is known. In vitreal experiments, it was assumed that Henry's constant was the same as that for the buffer. This may be true, but it was never measured. Most notably, Henry's constant for lenses has never been measured. It is important for this to be determined, though, since many experiments discussed herein require probing of both the lens and the vitreous, and it is desirable to ensure that any measured pO2 differences reflect real concentration differences.

Of the methods available, the only one appropriate for an intact lens is a physical method called the static headspace method (Allen et al., Determination of Henry's Law Constants by equilibrium partitioning in a closed system using a new in situ optical absorbance method. Environ. Toxicology and Chem., 17:1216-221, 1998). Essentially, the sample is placed in an airtight chamber, and degassed. Air is then allowed into the chamber; the chamber is sealed, and oxygen is allowed be taken up by the material until equilibrium is reached. Since the volumes of sample and air are known, the amount taken up by the sample is then known. From this, the actual solubility is determined; in turn, this is used to calculate Henry's constant, in accordance with Henry's law.

The probing device depicted in FIG. 10 has been fabricated, and can be made in any size. The one shown will fit a whole calf or rabbit lens. The sample is de-aerated, either with bubbling argon or under vacuum using the side arms. Air is then allowed in through the same inlets, and the device is sealed. Oxygen changes are monitored with a probe through the septum. Prior to the experiment, the device is calibrated so that the volume of the lens is known. This can be done by adding a known amount of buffer to a specific volume mark.

The samples for which Henry's constant is determined include vitreous, increasing concentrations of alpha crystallin, and whole intact lenses. Rat lenses are used to increase surface area and decrease the time it takes to reach equilibrium. Lenses present a special problem, since they actively metabolize oxygen. To circumvent this, buffer may contain KCN, to kill the epithelial cells. This method was used in early experiments investigating lenticular oxygen metabolism (Yorio et al., Aerobic and anaerobic metabolism of the crystalline lens of a poikilotherm; the toad Bufo marinus. Comparative Biochemistry and Physiology, 62:123-26, 1979; Lou and Kinoshita, Control of lens glycolysis. Biochim. Biophys. Acta, 141:547-59, 1967).

Diffusion and Concentration

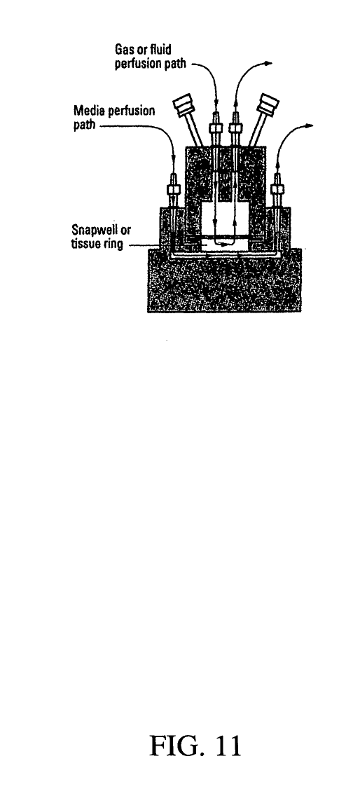

To verify the above results and the relative rates of oxygen diffusion through a lens, a new technique may be used to measure non-steady-state diffusion (Lamers-Lemmers et al., Non-steady-state O₂ diffusion in metamyoglobin solutions studied in a diffusion chamber. Biochemical and Biophysical Res. Commun., 276:773-78, 2000), i.e., the diffusion of oxygen through samples using a diffusion chamber obtained from Harvard Apparatus, Inc. (Holliston, Mass.). Essentially, this technique consists of a sample isolated between two chambers. Both are evacuated with argon until the sample comes to equilibrium; the top chamber is then equilibrated with an air sample, while the bottom is closed. The oxygen content in both chambers is monitored continuously with probes. The changes in oxygen partial pressure (pO₂) are analyzed to determine the rate of oxygen diffusion through that sample (m²×s⁻¹), and the amount of oxygen diffusing through that layer (in mol×m⁻¹×kP⁻¹). From that, oxygen solubility is determined from the following relationship: amount of O₂ diffusing=rate×solubility. For these studies, a modified horizontal diffusion chamber (depicted in FIG. 11), designed to isolate the two chambers, is used.

In accordance with this methodology, optodes are placed in both chambers; a hole may have to be drilled in the lower chamber. Samples are placed in a Snapwell device, for which these chambers were designed. The studied samples are the same as those studied for Henry's constant, except that lens slices, as opposed to whole lenses, are used, in accordance with techniques well known in the art (Dillon et al, Transmission characteristics of the lens of the primate eye. IOVS Invest. Ophthalmol. Vis. Sci., 41:1454-459, 2000).

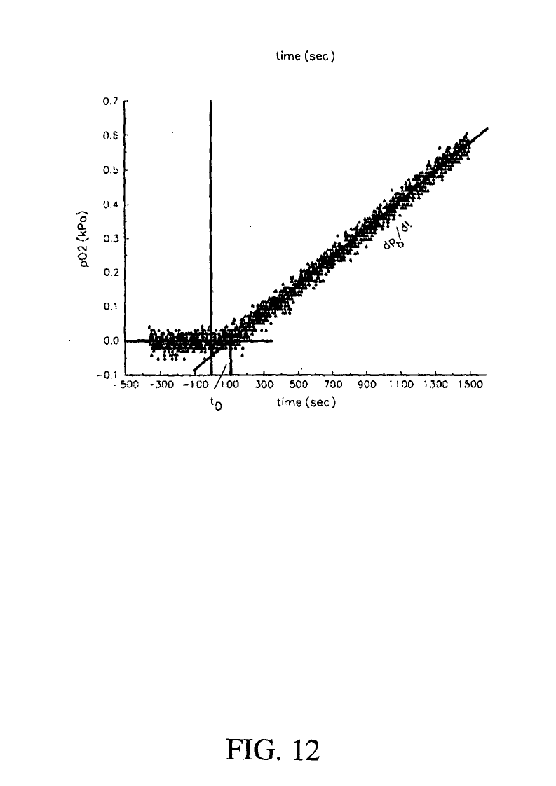

FIG. 12 depicts, on the right, the oxygen tension at the bottom of the chamber. After a delay time (tₒ), oxygen diffuses through the sample, and is detected in the lower chamber. Thereafter, the oxygen content increases linearly. The line is fitted to a straight line having a slope dpₒ/dt. After correcting for diffusion through the membrane, and comparing to standards, tₒ will represent the rate of diffusion through the sample, and dpₒ/dt may be used to calculate the oxygen permeability. From those values, oxygen concentration may be calculated (Lamers-Lemmers et al., Non-steady-state O₂ diffusion in metamyoglobin solutions studied in a diffusion chamber. Biochemical and Biophysical Res. Commun., 276:773-78, 2000).

In order to overcome the problems associated with ascertaining oxygen content when the samples are degassed, lenses or lens slices are placed in the Henry-constant chamber with a stirring bar. After degassing for various periods of time, de-aerated buffer is introduced, the lenses are homogenized, and the amount of oxygen is assessed. Since the accuracy of this method, when using concentrated alpha crystallin solutions, is known, the amount of residual oxygen can be accurately determined.

Tissue Culture

Lens organ culture has been utilized for more than 75 years by biologists attempting to understand the physiology of lens tissue (Bakker, A., Die regeneration der verwundeten linsenkapsel von kaninchenslinses in der durchströmungskultur. von Graefes Arch. Ophthalmol., 136:333-40, 1937; Kinsey et al., Studies on the crystalline lens VI. Mitotic activity in the epithelia of lenses cultured in various media. Am. J. Ophthalmol., 40:216, 1955). Many types of growth media, supplements, and surgical/culturing approaches have been described, and a variety of animal lenses are routinely utilized (Chylack and Kinoshita, The interaction of the lens and the vitreous II. The influence of the vitreous on lens trauma water and electrolyte balance and osmotic stress. Exp. Eye Res. 15:61-69, 1973; Korte et al., A comparison of two buffering systems for lens organ culture. Ophthalmic Res., 14:265-68, 1982). The rabbit lens is a useful model for such studies, because of its size and well-characterized biochemical and physiological properties (Reddan et al., Regional differences in the distribution of catalyses in the epithelium of the ocular lens. Cell. Mol. Biol., 42:209-19, 1975; Reddan et al., Induction of mitosis in the cultured rabbit lens initiated by the addition of insulin to medium KEI-4. Exp. Eye Res., 20:45-61, 1975; Fischbarg et al., Transport of fluid by lens epithelium. Am. J. Physiol., 276:C548-C557, 1999).

A number of cellular, biochemical, and molecular biological methodologies are available to assess cell viability and function. They range from simple morphological examinations of cell viability (e.g., Trypan blue exclusion), cell death (various dye-exclusion-based live/dead assays, including Hoechst, propidium iodine, and acridine orange), mitochondrial function (e.g., MTT tetrazolium colorimetric assays), and apoptotically-induced DNA damage (e.g., TUNEL), to rapid biological assays for DNA and RNA synthesis (e.g., [³H]thymidine and uridine incorporation), active transport (e.g., ¹⁴choline and ⁸⁶Rb uptake), and membrane permeability (e.g., ⁸⁶Rb efflux), and to more complex assays for DNA damage (e.g., alkaline elution and single cell gel assay), intracellular ATP and NAD levels, and enzyme activity (e.g., GPD). A number of investigators have described growth media suitable for maintenance of lens transparency, metabolism, and physiology. Some of the more experimentally-useful systems are cell-culture-medium-based, are often Hepes-buffered, contain serum, and may be supplemented with additional growth-promoting components. In the present studies, buffered medium is used to reproduce, as closely as possible, the physiological and physiochemical properties of rabbit aqueous (Riley, M. V., The chemistry of the aqueous humor. In Biochemistry of the Eye, Anderson, R. E., ed. (San Francisco: American Academy of Ophthalmology, 1983), pp. 79-95.

The following experiments are performed in normal organ culture, because oxygen tension can be readily be controlled. These experiments will provide the necessary preliminary to full-scale rabbit experiments, since they provide the approximate period of time in which to run and terminate the in vivo experiments.

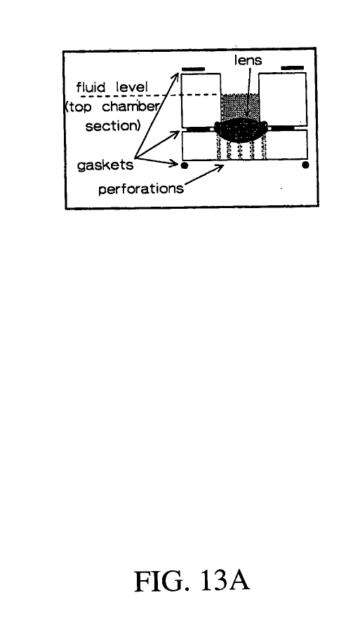

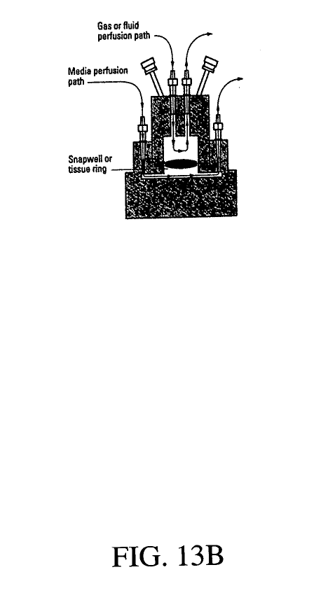

Because the lens is asymmetrical, a device has been developed to account for the asymmetry. FIG. 13A depicts a device which isolates the anterior (top) from the posterior portion of the lens, with the use of gaskets. This device may be used with the probe device described above, in which the chamber has been modified, as shown in FIG. 13B. Both the anterior and posterior lenses have segregated inlets and outlets for separate perfusion, and both the chambers are fitted with individual oxygen probes.

Initial experiments may be performed on lenses in culture, to determine the effect of higher environmental oxygen tension and damaged epithelia on oxygen diffusion into the lens, as described herein. Once the chamber is developed, the following experiments may then be performed.

1. The chamber is tested for gas leakage by filling the posterior chamber with argon-saturated media, with the other chamber in 5% oxygen (average aqueous oxygen tension). The ports are sealed, and oxygen content is followed for the expected time course of the experiment (1-2 days). Any leakage around the gaskets will occur quickly. However, over the time course of the experiment, leakage may also occur, not around the gasket, but through the lens. This will be evident from the following determinations: (a) if it occurs through the lens, there will be a lag time, followed by an almost-constant rise in oxygen tension, as described below; and (b) the oxygen content of the lens may be probed, as indicated in FIG. 1A. Any deviations from this profile may be taken as an indication of oxygen leakage through the lens, thereby providing accurate data for the experiments hereinafter described.

2. Lenses may be maintained for various periods of time, perfused with artificial aqueous or other media. The anterior chamber may be held at 5% oxygen, and the posterior at 2%. This may be accomplished by changing the media periodically, or by slowly pumping media through the chamber. After various periods of time, the lenses may be checked for oxygen content and epithelial cell viability (as described herein). Both tests may be performed on the same lens, since the oxygen electrode is only 60-100 μm in diameter.

3. Once the viability of the tissue culture is established, the posterior chamber may be perfused with media at 21% oxygen, or with a tamponade at 21% oxygen. After various periods of time, the experiment may be aborted, and the lens may be tested for any increases in oxygen tension.

4. In general, it has been found that there is an age-dependant decrease in enzyme activity in the lens (Hockwin et al., Influence of age on enzyme activities of lenses. Ophthalmologica, 150:187-95, 1965). These enzymes include those involved in energy metabolism, such as glucose-6-P-dehydrogenase and glyceraldehyde phosphate dehydrogenase. It has also been found that there is a decrease in activity in cataractous lenses, when age matched (Hockwin and Orhloff, Enzymes in normal, aging and cataractous lenses. In Molecular and Cellular Biology of the Eye, Bloemendal, H., ed. (New York: John Wiley & Sons, 1981), pp. 367-414; Young, R. W., Age-related deterioration of the lens. In Age-Related Cataract (Oxford: Oxford University Press, 1991), pp. 33-56. The decreases described above will presumably reduce the lenses' utilization of oxygen. With the same oxygen tension available from the aqueous, this should result in an increase in oxygen in the lens, and, in accordance with the discoveries described herein, nuclear cataract formation should then ensue.

To test this hypothesis of cataract formation, the epithelial layer may be damaged by increasing degrees with either hydrogen peroxide (Kleiman et al., Hydrogen peroxide-induced DNA damage in bovine lens epithelial cells. Mutation Res., 240:35-45, 1990) or photosensitized oxidation (Spector et al., A brief photochemically induced oxidative insult causes irreversible lens damage and cataract II. Mechanism of Action. Exp. Eye Res., 60:483-92, 1995a). The lenses may then be tested for epithelial cell viability, including Trypan blue exclusion, active transport, and GPD activity. Contralateral lenses, which are treated in the same manner, may be placed in the chamber, and subjected to normal oxygen tensions found in the aqueous and vitreous. The experiment may be aborted at various periods of time, and oxygen tension may be assessed throughout the lens. Any increases in oxygen are taken as evidence that the above-described process can occur in humans.

5. In cases 3 and 4 above, the lens is supplemented with ascorbic acid prior to and during the experiment, to determine the putative effect of ascorbic acid in the reduction of oxygen tension. In addition, for case 4, oxygen tension is decreased in the anterior chamber, in an attempt to decrease oxygen uptake in the lens. The results of such experiments lead to the conclusion that a simple prophylactic method to reduce the incidence of nuclear cataracts includes a reduction in the amount of oxygen available to the lens.

Although it has been demonstrated (McLaren et al., Measuring oxygen tension in the anterior chamber of rabbits. Inves. Ophthalmol. Vis. Sci., 39:1899-909, 1998) that the use of a hard contact lens in a rabbit almost completely reduces the amount of oxygen in the aqueous, one embodiment of the present invention is based on the discovery that a contact lens that is semi-permeable—allowing enough oxygen for the cornea and epithelial of the lens, but not enough to overwhelm the lens's defenses—can be used to prevent nuclear cataracts. Following a vitrectomy, the vitreous may be replaced by various solutions, including atmospheric oxygen or a tamponade with air present. The length of time that it takes for oxygen to reduce to normal vitreal levels is determined, as is the length of time sufficient to lead to increased oxygen tensions within the lens. In accordance with this method, the putative chemical processes involved in the reduction of oxygen tension in the vitreous may be investigated by performing the following experiments on rabbits:

1. The vitreous is replaced by standard BSS solution. Under anesthesia, the oxygen tension of the vitreous body is continuously sampled for 5-6 h. Periodically, vitreal samples are taken for HPLC analysis. After 5-6 h, the oxygen tension in the lens is analyzed by inserting an oxygen electrode through therein. The rabbit is then euthanized, and the lenses excised and stored at −70° C. for HPLC analysis. The second eye may act as a control.

Additionally, a second group of animals may undergo the same operation, to study long-term effects of the oxygen. After the vitrectomy and delays of 1 day to 4 weeks (approximately 4 groups—1:1 day; 2:1 week; 3:2 weeks; and 4:4 weeks), the animals are again placed under general anesthesia, and oxygen is measured in the vitreous and lens. Again, the rabbits are euthanized, and the lenses excised and stored at −70° C. Generally, three rabbits per point are necessary for reasonably accurate results.

2. After basal levels of oxygen uptake with time are determined, four additional experiments are performed. The same procedures are utilized, along with the following vitreous replacement solutions: (a) de-aerated BSS; (b) BSS saturated with 100% oxygen; (c) BSS with ascorbic acid supplementation; and (d) a tamponade consisting of perfluoropropane with 21% oxygen. Oxygen tension at various time points is assessed, vitreal samples are taken for HPLC, and the lenses are stored for HPLC analysis.

The first experiment results in reduced oxygen tension in the lens, which has an immediate extension to human vitrectomies, where vitreal replacements with reduced oxygen may be part of a prospective study. Tamponade-based vitrectomies are thought to cause nuclear cataracts faster than non-tamponade vitrectomies, due to the raid formation of a PSC. This, in turn, leads to a nuclear cataract. It has been suggested that this results from the interruption of nutrient flow (Hsuan et al., Posterior subcapsular and nuclear cataract after vitrectomy. J. Cataract Refract. Surg., 27:437-44, 2001). However, another hypothesis, as set forth herein, is that both types of nuclear cataracts (BSS+ and tamponade) are caused by increased oxygen. In the case of a gas, as in the tamponade, oxygen will diffuse some 4 magnitudes faster than in buffer. Therefore, it will encounter the lens many more times as a gas, and have a much greater chance of entering the lens.

3. All vitreous and lens samples taken in the above experiments are analyzed by HPLC, with electroanalytical detection for reducible components (Rose and Bode, Analysis of water-soluble antioxidants by high-pressure liquid chromatography. Br. Biophys. J., 306:101-05, 1995; Rose et al., Properties of electrochemically active components in mammalian vitreous humor. Exp. Eye Res., 64:807-12, 1997; Richer and Rose, Water soluble antioxidants in mammalian aqueous humor: interaction with UV B and hydrogen peroxide. Vision Res., 38:2881-88, 1998). In addition, levels of hydrogen peroxide are assessed (Spector and Wang, The aqueous humor is capable of generating and degrading H₂O₂. Inves. Ophthalmol. Vis. Sci., 39:1188-97, 1998).

From the foregoing studies, the following is determined:

1. By following the increasing levels of tyrosine and other aqueous components in the vitreous cavity, an accurate assessment may be made of the actual amount of time it takes for the aqueous to supplant BSS. Although this process is thought to be “fast”, it has not previously been measured kinetically, to the inventor's knowledge. A level of 100 μM tyrosine (Richer and Rose, Water soluble antioxidants in mammalian aqueous humor: interaction with UV B and hydrogen peroxide. Vision Res., 38:2881-88, 1998) may be taken as complete aqueous replacement of the vitreous cavity.

2. The levels of oxygen are compared to the levels of aqueous in the vitreous cavity, to assess the relative ability to decrease oxygen tension. These are then compared to experiments where BSS is supplemented with ascorbic acid and/or degassed.

3. If a chemical process involving ascorbic acid is reducing oxygen tension, then hydrogen peroxide is likely produced as a by-product (Eaton, J. W., Is the lens canned? Free Radic. Biol. Med., 11(2):207-13, 1991). Therefore, the concentration of hydrogen peroxide is assessed (Spector et al., The aqueous humor is capable of generating and degrading H₂O₂. Inves. Ophthalmol. Vis. Sci., 39:1188-97, 1998), and compared to levels of ascorbic acid and oxygen. A direct correlation between hydrogen peroxide and ascorbic acid, and an inverse correlation between those two compounds and oxygen, is taken as evidence that such processes are occurring in vivo.

4. It has been demonstrated that, during cataractogenesis, ascorbic acid and GSH decrease in lenses. Thus, correlations are made between increases in oxygen tension, and decreases in the two anti-oxidants, in lenses taken from these studies. These correlations are ascertained by sectioning frozen lenses, assessing levels of anti-oxidants in each section by HPLC, and comparing those results with any increases in oxygen tension detected by probing the lenses. Controls are the contralateral eyes.

Comparisons between anti-oxidant levels and oxygen tension increases in lenses for all four experiments will demonstrate that oxygen per se is a plausible oxidant, thereby explaining senile nuclear cataracts. This means that increases in oxygen tension in vitreal replacements should result in increased oxygen and decreased anti-oxidants in the lens. These may also be modulated by ascorbic acid supplementation.

Biological Implications

The experiments described above give very basic information concerning the concentration of, diffusion of, and the environmental effects on oxygen tension in the mammalian lens. In many cases, the experiments are interdependent. For example, vitrectomized rabbit experiments allow determination of the length of time for oxygen tension to decrease to pre-vitrectomized levels, and the rate at which this occurs. This guides the tissue culture experiments, where the posterior segment is perfused with increased oxygen tension. In that experiment, oxygen tension is decreased at the same rate that occurs in vivo. Conversely, the rate of increase in oxygen tension in the perfused lens guides the determination of the length of time needed to detect increases in oxygen in the in vivo experiments. In EPR experiments, one can only obtain a diffusion-concentration product for oxygen. However, the experiments described herein allow one to obtain oxygen concentration for lens slices. Therefore, a combination of the two experiments will allow diffusion in very small samples to be ascertained.

The biological implications for the studies described herein, and their results, are substantial. A major parameter in the etiology of nuclear cataracts in humans is an increase in oxygen tension in the nucleus of the lens; that oxygen tension is mediated by the environment around the lens, and the health of the epithelial layer. Oxygen in the environment of the lens is, in turn, mediated by oxygen flow across the cornea, retina metabolism, and reactions involving ascorbic acid. The experiments described herein provide evidence of these contentions.

### Example 4

### Measurement of Oxygen Tension in the Rabbit Eye Before and After Surgery

A total of 26 Harlan rabbits (3.5-5.3 kg; 6 months old) were used in this Example. The animals were anesthetized with an intramuscular injection of Xylazine (5 mg/kg) and Ketamine (35 mg/kg). The pupil was dilated by installing cyclopentolate hydrochloride (1%) and phenylephrine hydrochloride (10%) topically. A sclerotomy was made 6 mm posterior to the limbus, using a 23-gauge blade. The fiber-optic oxygen sensor (optode) was placed through the sclerotomy into the vitreous cavity, and was correctly positioned, under direct observation through the operation microscope, using coaxial light and a flat corneal contact lens. The oxygen probe was stabilized using a micromanipulator to ascertain the exact position of the probe within the eye, when needed. All animals were treated in accordance with the ARVO Statement for the use of animals in ophthalmic and vision research.

Oxygen Tension Measurement

The oxygen measurements were done using a commercially-available fiber-optic oxygen sensor system (FOXY Fiber Optic Oxygen Sensor systems, Ocean Optics Inc., USA), which is a spectrometer-coupled chemical sensor for quantitative measurements of dissolved and gaseous oxygen pressure. The principle of the measurement technique is based on the quenching of fluorescence by oxygen. In this case, the fluorescence of a ruthenium complex is used to measure the partial pressure of oxygen. The ruthenium complex is trapped in a sol-gel matrix, at the distal end of an optical fiber. The signals are carried through the optical fiber to the spectrometer, converted to digital data by an A/D converter, and displayed by a PC.

The fiber-optic oxygen sensor system does not consume oxygen; therefore, the movement of sample or sensor will not affect the final reading. The probe used in this Example—a modified version of the FOXY-AF model—was especially designed for these experiments by the inventor (FIG. 10). It has a reinforced anterior portion which prevents bending, thereby preventing falsification of the measurement. The tip of the probe (which is 300 μm in diameter) has a silicone overcoat to exclude ambient light and to improve chemical resistance, allowing for continuous contact with the sample. However, the overcoat slows the response time.

In the present Example, the response time for vitreous and lens measurements was about 2-5 min. The operating software was OOI Sensors. Prior to the measurements, the sensor was calibrated in water at 39° C., equilibrated to 100% argon (0 mmHg pO₂) and to room air (20.8% of 760 mmHg pO₂), respectively. The system has an accuracy of 1% of full range for 0-20%. At the end of each experiment, the calibration was repeated to control the stability of the equipment.

Vitreal Measurement

Mounted on a micromanipulator, the fiber-optic probe was placed through the sclerotomy into the vitreous cavity to measure the vitreous oxygen tension in 6-8 predefined positions within the vitreous cavity. FIG. 14 presents the raw data from such an experiment. Point number 5 (FIG. 14) is the position approximately 0.5 mm in front of the retina. For position number 6 (i.e., the surface of inner retina), the probe was advanced toward the retina until a subtle concave mirror effect on the retina could be seen through the operating microscope. The tip of the probe was placed away from any main retinal vessels.

Measurement in the Lens and Anterior Chamber

Lens measurements were performed at the post-vitrectomy follow-up examination, in the operated eyes, the control eyes, and a small series of control rabbits which did not undergo surgery. The oxygen probe was made of aluminum, with a diameter of 300 μm; therefore, it was reasonably flexible with a sharp tip. The probe could be easily inserted into the lens through the posterior capsule. The oxygen measurements were first performed in the central vitreous and in the anterior vitreous body close to the lens, and then in the posterior part of the lens, in the lens center, in the anterior part of the lens, and below the anterior capsule. To measure the oxygen tension in the aqueous humor, a tunnel incision was made at the limbus, through which the fiber-optic probe was carefully inserted into the middle of the anterior chamber in order to avoid loss of the aqueous humor.

Vitrectomy

Prior to vitrectomy, vitreous measurements were taken at different positions in the vitreous cavity: anterior, central, posterior, and pre-retinal. Thereafter, a vitrectomy was performed, as previously described (Abrams et al., An improved method for practice vitrectomy. Arch. Ophthalmol., 96(3):521-25, 1978), without cryotherapy. Because the rabbit has a small pars plana and a large lens relative to the size of the eye, the sclerotomy was made 5-6 mm posterior to the limbus, to ensure free movement of the measurement tool. BSS (Alcon) was used as infusion during vitrectomy, and was stored at 39° C. After vitrectomy was completed, an optode was placed immediately through the sclerotomy, and into the center of vitreous cavity, to monitor oxygen changes in BSS. A pre-placed suture was created to prevent leakage. At the end of the measurements, all sutures were closed, and the eyes were treated with bactriacin ointment and cyclogel eye drops.

Following vitrectomy, the rabbits were re-anesthetized, and oxygen measurements in the vitreous cavity and the lens were obtained at 1 week, 2 weeks, 4 weeks, and 8 weeks. Parallel measurements in the fellow eye served as controls. Eyes with surgical complications, such as retinal detachment (1 eye), cataract due to a “lens touch” (1 eye), and hemorrhage (1 eye), were excluded from follow-up examinations. All animals were sacrificed after the second procedure.

Discussed below are results obtained by the inventor in connection with the experiments of Example 4:

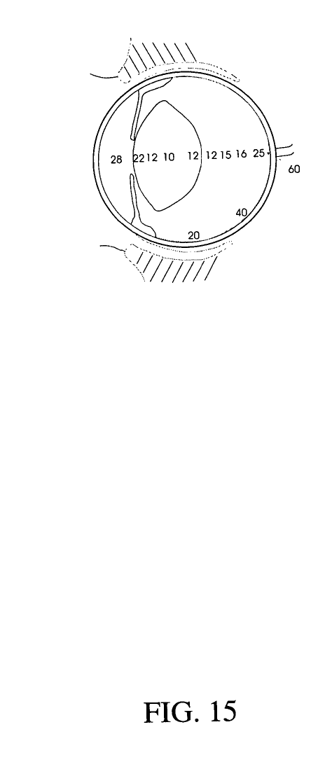

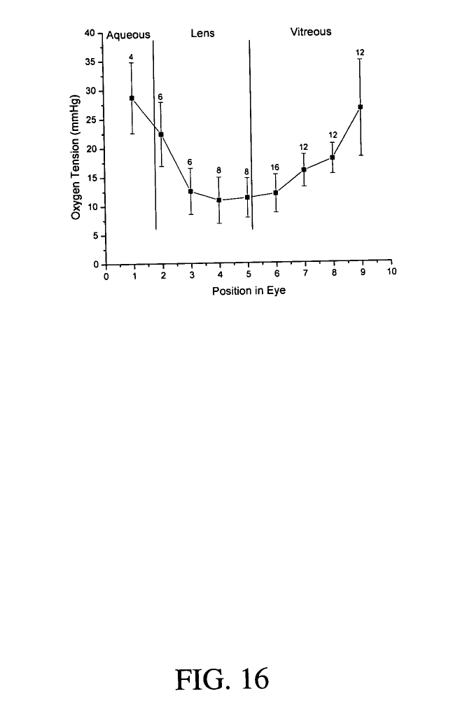

FIGS. 15 and 16 set forth results of measurements taken in normal rabbit eyes. Oxygen tension within the rabbit globe was asymmetrical, with the lowest pO₂ measurement in or near the nucleus of the lens (9.4 mmHg±1.2). From the anterior to the posterior of the lens, there was a fairly steep gradient. The oxygen tension directly below the lens epithelium was approximately 2 times higher than that in the center or posterior part of the lens. The region near the posterior capsule had an oxygen tension close to the values of the central vitreous directly behind it (10 mmHg±0.4). The highest pO₂ within the posterior compartment of the eye was measured close to the retinal surface (40-60 mmHg), depending upon neighboring large vessels. The tension dropped off rapidly to 20 mmHg, approximately 0.5 mm from the retina. From that position to the posterior surface of the lens, there was a shallow gradient of decreasing pO₂ (FIGS. 15 and 16).

Measurements taken in the anterior vitreous, but close to the retina, were also higher (approximately 20 mmHg) than the values obtained in the central vitreous (10-15 mmHg). FIG. 15 shows an original pO₂ profile measured in a rabbit anterior vitreous and lens. The aqueous humor oxygen tension, measured in the center of the anterior chamber, was 28.7±6.1 mmHg (FIG. 16).

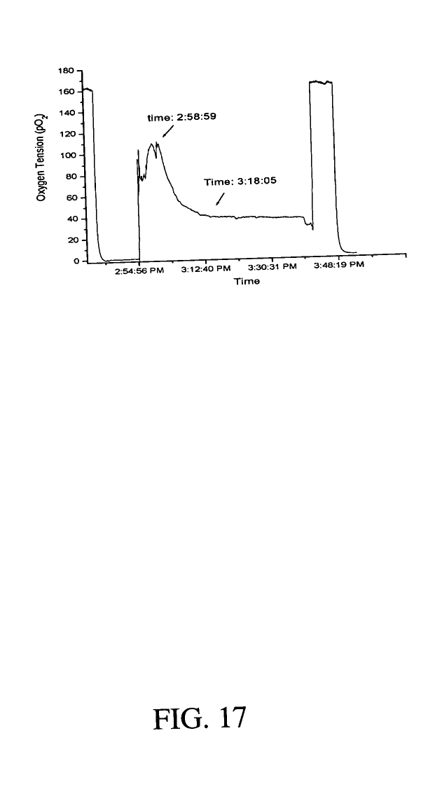

Immediately following vitrectomy, the pO₂ in the BSS replacement varied from 90 to 140 mmHg. Over approximately 30 min, it decreased to “steady-state” levels that were 2-3 times that of the normal vitreous (FIG. 17), resulting in oxygen values of 28.9±12.2 mmHg (6 eyes) (Table 1). The surprising variability in the initial oxygen tension reading, just after completion of the surgery, was most likely due to equilibration of the BSS with room air. The inventor discovered that pO₂ in BSS was roughly 70 mmHg, directly after opening of the bottle (due to autoclaving in manufacture). As the BSS was allowed to equilibrate with room air through the irrigation system, there was a significant increase in BSS oxygen tension.

TABLE 1
Overview of the oxygen measurements taken in the middle of the vitreous
cavity immediately following vitrectomy, and the time elapsed until the
values levelled off.

| | Initial pO₂ post | Leveled off pO₂ | Time |
|---|---|---|---|
| Case No. | surgery (mmHg) | (mmHg) | (min) |
| 1 | 140.6 | 33.7 | 32 |
| 2 | 110.2 | 38.2 | 19 |
| 3 | 119.3 | 11.2 | 30 |
| 4 | 136.1 | 42.1 | 45 |
| 5 | 88.2 | 16.0 | 20 |
| 6 | 121.6 | 30.6 | 12 |
| Mean ± SD | 119.3 ± 18.9 | 28.9 ± 12.2 | 26.3 ± 11.8 |

To quantify these findings, a separate experiment was conducted. BSS solution was allowed to drop at a rate of about 120 drops/min, with an equal volume of air bubbled into the bottle. After 10 min, the pO₂ increased to 110 mmHg; after 50 min, the pO₂ increased to 160 mmHg. Therefore, it can be assumed that the lens is exposed to a high and variable level of oxygen (at least 10 times higher than normal) during vitrectomy, depending on the time of surgery.

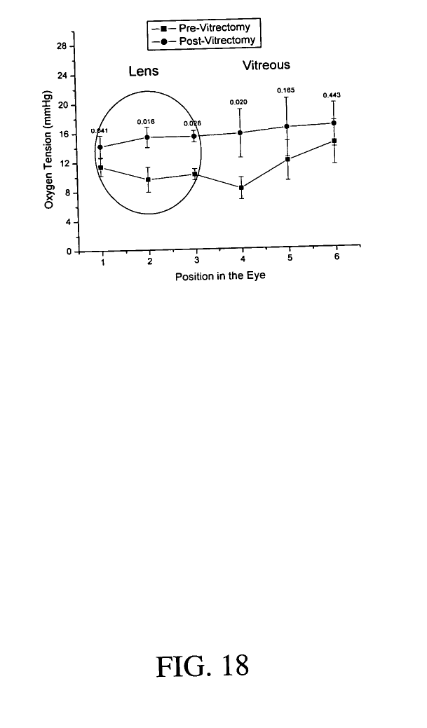

To follow long-term oxygen changes after vitrectomy, measurements in the vitreous cavity and the lens were taken at 2 weeks, 4 weeks, and 8 weeks. Except for values obtained close to the retina, oxygen tension was invariably greater for the operated eyes, when compared to the controls, even after 8 weeks (and in one case after 12 weeks). The greatest difference, however, appeared to be after 2 weeks. At 2 weeks post-vitrectomy, the pO₂ values in the center, in the posterior lens, and directly behind the lens, were 2-3 times as high as in the control eye (p<0.05). In addition, there was no longer a gradient in the vitreous cavity, except close to the retina (FIG. 18). To get a sense of the overall changes in oxygen tension that occur in the eye after a vitrectomy, the data was combined and plotted in (FIG. 18).

Eight weeks after vitrectomy, pO₂ levels in the lens were decreased, but still remained higher than in the normal eye (about 20% higher than in the control eye). Again, the previously-described pO₂ gradient in the vitreous was not detectable at any of the follow-up examinations.

While the invention has been described and illustrated in connection with preferred embodiments, many variations and modifications as will be evident to those skilled in this art may be made without departing from the spirit and scope of the invention, and the invention is thus not to be limited to the precise details of methodology or construction set forth above as such variations and modification are intended to be included within the scope of the invention.

What is claimed is:

1. A method for protecting against cataract development in a subject, during a vitreous replacement, comprising use of a vitreous replacement solution having a low oxygen concentration.

2. The method of claim 1, wherein the oxygen concentration of the low-oxygen-concentration solution is between about 0% and about 2%.

3. The method of claim 2, wherein the oxygen concentration is about 0%.

4. The method of claim 1, wherein the low-oxygen-concentration solution includes reduced glutathione and ascorbic acid.

5. The method of claim 1, wherein the low-oxygen-concentration solution includes reduced glutathione.

6. The method of claim 5, wherein the glutathione in the solution has a concentration between about 0.01 mM and about 10 mM.

7. The method of claim 6, wherein the glutathione concentration is between about 0.1 mM and about 2 mM.

8. The method of claim 7, wherein the glutathione concentration is about 1 mM.

9. The method of claim 1, wherein the low-oxygen-concentration solution is an initial vitreous replacement solution from which at least a portion of the oxygen has been removed.

10. The method of claim 9, wherein the at least a portion of the oxygen is removed by subjecting the initial solution to at least a partial vacuum.

11. The method of claim 10, wherein the initial solution is subjected to the at least a partial vacuum for about 10 minutes to about 15 minutes.

12. The method of claim 9, wherein the at least a portion of the oxygen is removed by introducing an essentially-oxygen-free gas into the initial solution.

13. The method of claim 12, wherein the essentially-oxygen-free gas is an inert gas.

14. The method of claim 12, wherein the essentially-oxygen-free gas is a noble gas.

15. The method of claim 12, wherein the essentially-oxygen-free gas is nitrogen gas.

16. The method of claim 12, wherein the essentially-oxygen-free gas is introduced into the initial solution by bubbling the gas through the initial solution, thereby producing a low-oxygen-concentration solution.

17. The method of claim 16, wherein the gas is bubbled through the initial solution for about 10 minutes immediately prior to introduction of the low-oxygen-concentration solution into an eye of a subject.

18. The method of claim 1, wherein the low-oxygen-concentration solution includes ascorbic acid.

19. The method of claim 18, wherein the ascorbic acid in the solution has a concentration that is sufficiently high to protect against cataract development in a subject.

20. The method of claim 18, wherein the ascorbic acid concentration is between about 0 mM and about 10 mM.

21. The method of claim 20, wherein the ascorbic acid concentration is between about 0.5 mM and about 5 mM.

22. The method of claim 21, wherein the ascorbic acid concentration is between about 1 mM and about 3 mM.

23. The method of claim 22, wherein the ascorbic acid concentration is about 2 mM.

24. Use of a vitreous replacement solution having a low-oxygen concentration during a vitrectomy, wherein the low-oxygen-concentration solution is produced by removing at least a portion of the oxygen from an initial vitreous replacement solution.

25. The use recited in claim 24, wherein the low-oxygen-concentration solution includes reduced glutathione.

26. A low-oxygen-concentration vitreous replacement solution for use in vitrectomies, wherein the low-oxygen-concentration solution is an initial vitreous replacement solution from which at least a portion of the oxygen has been removed.

27. The low-oxygen-concentration solution of claim 26, which includes reduced glutathione.

28. The low-oxygen-concentration solution of claim 26, which includes ascorbic acid.

29. The low-oxygen-concentration solution of claim 26, which includes reduced glutathione and ascorbic acid.

30. A method for protecting against cataract development and/or for treating a cataract in a subject, comprising reducing oxygen concentration in a vitreous of the subject.
